(19) United States
(12) Patent Application Publication
(10) Pub. No.: US 2025/0170178 A1
(43) Pub. Date: May 29, 2025

(54) METHODS AND PRODUCTS FOR CULTURING T CELLS AND USES THEREOF

(71) Applicant: QUELL THERAPEUTICS LIMITED, London (GB)

(72) Inventor: Georgios ELEFTHERIADIS, London (GB)

(21) Appl. No.: 18/844,930

(22) PCT Filed: Mar. 15, 2023
(86) PCT No.: PCT/GB2023/050610

§ 371 Date: Sep. 6, 2024

(30) Foreign Application Priority Data
Mar. 22, 2022 (GR) 20220100253
Mar. 28, 2022 (GB) 2204328.5

(51) Int. Cl. A61K 35/17; A61K 40/11; A61K 40/22; A61K 40/31; A61K 40/41; C07K 14/47; C12N 5/00; C12N 5/0783

(52) U.S. Cl. CPC A61K 35/17; A61K 40/11; A61K 40/22; A61K 40/31; A61K 40/416; C07K 14/4702; C12N 5/0018; C12N 5/0637; C12N 2501/13

(57) ABSTRACT

The present invention provides methods of enhancing the persistence of regulatory T cells (Tregs), both in vitro and in vivo, and methods of reducing the expansion time of these cells in culture, by culturing the cells with one or more glial cell line-derived neurotrophic factor (GDNF) family ligands (GFLs).

### FIELD OF THE INVENTION

The present invention relates to methods of enhancing the persistence of regulatory T cells (Tregs), both in vitro and in vivo, and to methods of reducing the expansion time of these cells in culture, by culturing the cells with one or more glial cell line-derived neurotrophic factor (GDNF) family ligands (GFLs). The invention also provides a method of immunomodulation of a subject comprising formulating said cells, which have been cultured with one or more GFLs, into a medicament and administering said medicament to a subject. In addition, the invention provides a culture medium suitable for culturing said Treg cells, which comprises one or more GFLs.

### BACKGROUND TO THE INVENTION

In recent years, there has been increasing interest in using regulatory T cells (Tregs) in the clinical setting for adoptive cell therapy (ACT) to treat a range of different conditions. Tregs have been proposed for use in controlling undesired immune responses based on their immunosuppressive function. For example, Tregs have been used for the treatment of autoimmune or allergic diseases, for immunomodulation in transplantation, and to improve and/or prevent immune-mediated organ damage in inflammatory disorders. In addition, Tregs have been genetically engineered to express T cell receptors (TCRs) or chimeric antigen receptors (CARs) with new specificities, offering the advantage of providing targeted immune suppression. Various phase I trials have shown that Treg therapy is safe and efficacious and there are currently several phase II trials underway.

The main source of Tregs for therapeutic use is from a patient's own blood (drawn directly from a blood vessel or as a product of leukapheresis) or from umbilical cord blood. The number of Tregs from these sources is low and substantial numbers are required to effectively suppress the immune system. Therefore, ex-vivo expansion is necessary to obtain adequate numbers of Tregs for infusion into the patient. A typical isolation and expansion protocol that follows Good Manufacturing Practice (GMP) guidelines takes from approximately nine days up to twenty-one days and ensuring survival of the Tregs during this time is a challenge. It would be highly advantageous if survival and proliferation of the Tregs during the manufacturing process could be enhanced. This could reduce the amount of time needed to achieve a therapeutic dose of cells, so that the cells would be ready for more immediate use in the patient and would avoid prolonged processing, which can have a detrimental effect on Treg quality and persistence. In addition, enhancing proliferation and reducing the expansion time would reduce the costs associated with Treg cell production.

During ex vivo expansion, Tregs are typically cultured with the growth factor interleukin-2 (IL-2) which is essential for the homeostasis of these cells (generation, proliferation, survival), as well as for their suppressive function and phenotypic stability. In vitro and ex vivo expansion protocols typically require high concentrations of IL-2 resulting in high dependency on this cytokine for survival. Activated conventional T cells (Tcons) are the main source of IL-2 in vivo. Tregs cannot produce IL-2 and depend on paracrine access to IL-2 produced by Tcons in the microenvironment. The availability of IL-2 in vivo has a critical impact on the therapeutic effects of Tregs expanded ex vivo and transferred into patients. However, IL-2 can be lacking in the inflamed tissue microenvironment and in patients that have received immunosuppressive drugs, as these drugs reduce the availability of IL-2 produced from Tcon cells. Therefore, the ability of administered Tregs to persist in vivo may be affected by their dependency on IL-2 and low in vivo IL-2 levels.

As such, approaches to improve the ability of Tregs to proliferate, survive and function, both ex vivo and in subjects receiving ACT, are highly desirable.

Glial cell line-derived neurotrophic factor (GDNF) family ligands (GFLs) are a family of neurotrophic factors, which belong to the transforming growth factor-β (TGF-β) superfamily and that were originally found to effect the development and maintenance of neuronal cells. There are four ligands in the GDNF family, namely GDNF, neurturin (NRTN), artemin (ARTN) and persephin (PSPN). All four ligands have a similar structural conformation and all signal primarily through RET (Receptor Tyrosine Kinase) by first binding a class of proteins known as GDNF-family receptor-α (GFRα) receptors (Airaksinen and Saarma, Nature Reviews Neuroscience, 2002, Volume 3:383-394). It is known that the concentration of GFLs is altered in certain disease environments. For example, in amyotrophic lateral sclerosis (ALS), levels of GDNF are increased in muscles of mouse ALS models but decreased in serum (Stanga et al., Front Neurol., 2018; 9:384). Furthermore, neurotrophic factors have been shown to support neuronal survival and induction of their expression can prevent neurodegeneration.

### SUMMARY OF THE INVENTION

The present inventors have surprisingly found that culturing Tregs with a glial cell line-derived neurotrophic factor (GDNF) family ligand (GFL), for example GDNF, can significantly enhance their proliferation and survival ex vivo. Thus, when manufacturing Tregs for ACT, ex vivo expansion times can be significantly reduced by culturing the Tregs with one or more of these GFLs, in particular GDNF. In addition, the positive effects of these factors during the ex vivo expansion step may provide a survival advantage in vivo, as the administered cells have better viability and function at the point of administration, as compared to Tregs that have not been cultured in the presence of a GFL. Thus, these cells may persist longer in vivo, resulting in a better immunosuppressive effect overall.

The present inventors have discovered that the enhanced proliferation and survival effect of the GFLs is also seen in engineered Tregs, such as those which have been engineered to express a CAR and/or FOXP3.

Thus, the inventors have provided a solution to the problem associated with ex vivo proliferation, survival and expansion times, and in vivo persistence of Tregs, providing a path for producing Treg therapies that can be effectively used in the clinic.

Accordingly, the invention provides an ex vivo method of increasing the persistence of a regulatory T cell (Treg) or a population of Tregs, comprising the step of culturing the Treg or population of Tregs with one or more glial cell line-derived neurotrophic factor (GDNF) family ligands (GFLs). Alternatively viewed, the invention provides an ex vivo method of increasing the proliferation and/or survival of a regulatory T cell (Treg) or a population of Tregs, comprising the step of culturing the Treg or population of Tregs with one or more glial cell line-derived neurotrophic factor (GDNF) family ligands (GFLs). In one embodiment, the invention provides an ex vivo method of increasing the persistence of a regulatory T cell (Treg) or a population of Tregs, comprising the step of culturing the Treg or population of Tregs with GDNF. In an alternative embodiment, the invention provides an ex vivo method of increasing the proliferation and/or survival of a regulatory T cell (Treg) or a population of Tregs, comprising the step of culturing the Treg or population of Tregs with GDNF. Particularly the increase in persistence, proliferation or survival of the Treg or Treg population is as compared to a Treg or Treg population cultured under similar or the same conditions but without the presence of a GFL.

In another aspect, the invention provides an ex vivo method of reducing the expansion time of a regulatory T cell (Treg) or a population of Tregs, comprising the step of culturing the Treg or population of Tregs with one or more glial cell line-derived neurotrophic factor (GDNF) family ligands (GFLs). In one embodiment, the invention provides an ex vivo method of reducing the expansion time of a regulatory T cell (Treg) or a population of Tregs, comprising the step of culturing the Treg or population of Tregs with GDNF. Particularly, the reduction of expansion time of the Treg or Treg population is as compared to a Treg or Treg population cultured under similar or the same conditions but without the presence of a GFL.

In a further aspect, the invention provides a method of expanding a regulatory T cell (Treg) or a population of Tregs comprising the step of culturing said Treg or population of Tregs in cell culture media comprising one or more glial cell line-derived neurotrophic factor (GDNF) family ligands (GFLs). The one or more GFLs may be GDNF.

The invention also provides a method of immunomodulation of a subject in need thereof comprising the steps of:
(a) culturing a regulatory T cell (Treg) or population of Tregs ex vivo with one or more glial cell line-derived neurotrophic factor (GDNF) family ligands (GFLs);
(b) formulating the Treg or population of Tregs into a medicament; and
(c) administering the medicament to the subject.

The one or more GFLs may be GDNF.

Also encompassed is a method of producing an immunomodulatory medicament, the method comprising the steps of:
(a) culturing ex vivo a regulatory T cell (Treg) or population of Tregs with one or more glial cell line-derived neurotrophic factor (GDNF) family ligands (GFLs); and
(b) formulating the Treg or population of Tregs into a medicament.

The one or more GFLs may be GDNF.

In another aspect, the invention provides a method of improving survival of a regulatory T cell (Treg) or a population of Tregs after administration to a subject, comprising culturing the Treg or population of Tregs with one or more glial cell line-derived neurotrophic factor (GDNF) family ligands (GFLs) prior to administration of said cells. The one or more GFLs may be GDNF.

In a further aspect, the invention provides a culture medium suitable for culturing a regulatory T cell (Treg) or a population of Tregs comprising one or more glial cell-line derived neurotrophic factor (GDNF) family ligands (GLFs). In one embodiment, the invention provides a culture medium suitable for culturing a regulatory T cell (Treg) or a population of Tregs comprising GDNF.

The invention also provides the use of one or more glial cell line-derived neurotrophic factor (GDNF) family ligands (GFLs) for increasing the persistence of a regulatory T cell (Treg) or a population of Tregs. In one embodiment, the invention provides the use of GDNF for increasing the persistence of a regulatory T cell (Treg) or a population of Tregs. Alternatively viewed, the invention provides use of one or more glial cell line-derived neurotrophic factor (GDNF) family ligands (GFLs) for increasing the proliferation and/or survival of a regulatory T cell (Treg) or a population of Tregs. In one embodiment, the invention provides the use of GDNF for increasing the proliferation and/or survival of a regulatory T cell (Treg) or a population of Tregs. The increase in persistence, proliferation or survival of the Treg or population of Tregs may be after administration of the cell(s) to a subject.

The invention further provides the use of one or more glial cell line-derived neurotrophic factor (GDNF) family ligands (GFLs) for reducing the expansion time of a regulatory T cell (Treg) or a population of Tregs. In one embodiment, the invention provides the use of GDNF for reducing the expansion time of a regulatory T cell (Treg) or a population of Tregs.

Also encompassed is the use of one or more glial cell line-derived neurotrophic factor (GDNF) family ligands (GFLs) for culturing or expanding a regulatory T cell (Treg) or a population of Tregs in cell culture media. The one or more GFLs may be GDNF.

The invention also provides the use of one or more glial cell line-derived neurotrophic factor (GDNF) family ligands (GFLs) for producing a culture medium for culturing a regulatory T cell (Treg) or a population of Tregs. The one or more GFLs may be GDNF.

In yet another aspect, the invention provides a regulatory T cell (Treg) or population of Tregs for use in immunomodulation of a subject, wherein said Treg or population of Tregs have been cultured with one or more glial cell line-derived neurotrophic factor (GDNF) family ligands (GFLs) prior to administration to said subject. The one or more GFLs may be GDNF.

Also encompassed herein is a regulatory T cell (Treg) or population of Tregs obtained by a method of culturing said Treg or population of Tregs with one or more glial cell line-derived neurotrophic factor (GDNF) family ligands (GFLs). The one or more GFLs may be GDNF.

In another aspect, the invention provides a combination therapy or product comprising one or more glial cell line-derived neurotrophic factor (GDNF) family ligands (GLFs) and a medicament comprising regulatory T cells (Tregs), wherein the components of the combination therapy or product are for concurrent, or separate and sequential, use in immunomodulation of a subject. The one or more GFLs may be GDNF.

In yet a further aspect, the invention provides a kit comprising:
(i) one or more glial cell line-derived neurotrophic factor (GDNF) family ligands (GFLs); and
(ii) a culture medium suitable for culturing regulatory T cells (Tregs).

The one or more GFLs may be GDNF.

### DESCRIPTION OF FIGURES

FIG. 1 is a schematic of the assay used in Example 1 to assess the effect of culturing a mixed population of non-transduced (NTD) T cells (Treg and non-Treg cells) with various growth factors.

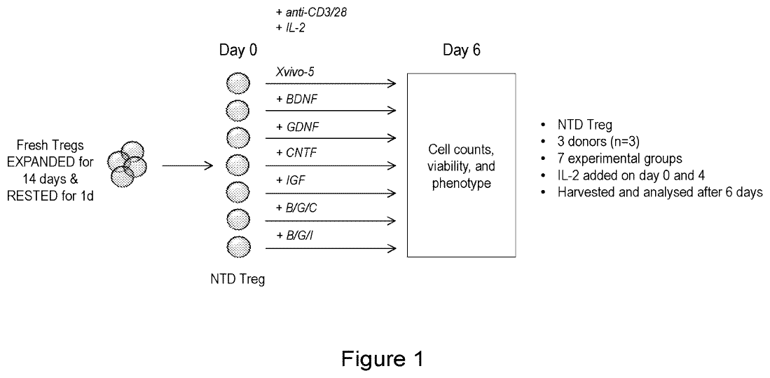

FIG. 2 shows the results of the assay described in FIG. 1. FIG. 2(a)—flow cytometric analysis identified the presence of a heterogeneous population of FOXP3+ and FOXP3− cells before (day 0) and after the end of the assay (day 6). FIG. 2(b) shows the gating strategy used to identify the counts of Live+CD4+ (FIG. 2(c)) and FOXP3+ and FOXP3− cells (FIGS. 2(d) and(e)). FIG. 2(c)—shows the number of Live+CD4+ cells in each treatment condition at day 6. GDNF significantly increases the number of Live+CD4+ cells in comparison to Xvivo-5 control (one-way ANOVA with Dunnett post-test, p=0.0327). FIGS. 2(d) and(e) show the same data (i.e., the number of live cells, which are either FOXP3+ or FOXP3−) with different statistical analysis. (d)—only in the GDNF treatment group is the number of FOXP3+ cells (white bars) significantly higher (two-way ANOVA with Fisher's post-test, p=0.0129) than the number of FOXP3− cells (black bars). (e)—only in the FOXP3+ group (white bars) is the number of cells significantly higher between the Xvivo-5 and GDNF treatments (two-way ANOVA with Dunnett post-test, p=0.0031).

FIG. 3 is a schematic of the assay used in Example 2 to assess the effect of culturing a highly pure population of Tregs (CAR-Tregs) with GDNF.

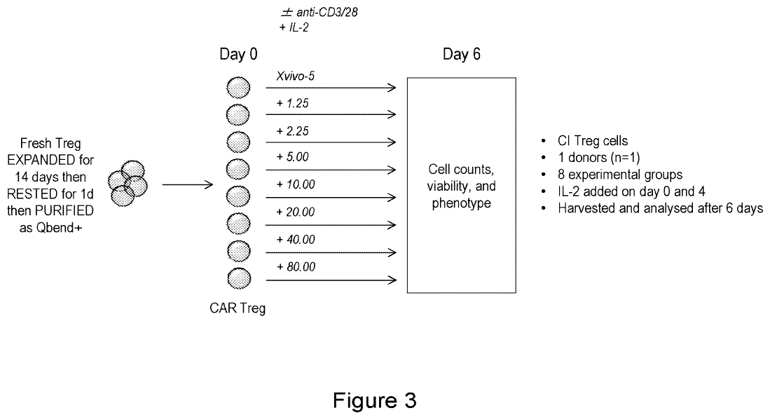

FIG. 4 shows the results of the assay described in FIG. 3. FIG. 4(a) shows representative flow cytometry staining showing the frequency of QBEND+ (CAR+) Treg cells before and after MACS. Magnetic purification of QBEND+ cells resulted in a highly pure population of CAR-Treg cells (98.6% QBEND+) on day 0. FIG. 4(b) and(c)—the frequency of QBEND+ (CAR+) and total FOXP3+ Treg cells is shown at rest (b) and at stimulation conditions (c) after the end of the assay on day 6. FIG. 4(d) and(e)—show the number of Live+CD4+ and FOXP3+ cells cultured with Xvivo-5 (control) and with different concentrations of GDNF on day 6, under resting (d) and stimulation conditions (e). GDNF enhances the number of CAR-Treg cells at rest (d) and at stimulation conditions (e), thereby resulting in a higher number of FOXP3+ CAR-Treg cells in comparison to Xvivo-5 control. During stimulation, GDNF acts in a concentration-dependent manner to enhance Treg persistence and 10 ng/ml of this factor yields an optimum effect.

FIG. 5 shows the results of the assay described in FIG. 3, but with only one concentration of GDNF (10 ng/ml). FIG. 5(a) shows representative histograms showing the counts of FOXP3+ cells at rest and at stimulation conditions on day 6. FIG. 5(b) shows that after stimulation, GDNF (10 ng/ml) significantly increases the number of Live+CD4+ cells (unpaired t-test, p=0.0002), thereby resulting in a significantly higher number of FOXP3+ CAR-Treg cells (unpaired t-test, p=0.0006) in comparison to Xvivo-5 control.

### DETAILED DESCRIPTION

The present invention provides, amongst other things, methods of increasing the persistence of a Treg or a population of Tregs comprising culturing the Treg or population of Tregs with one or more GFLs, such as GDNF. Alternatively viewed, the invention provides use of one or more GFLs, such as GDNF, for increasing the persistence of a Treg or population of Tregs.

The term “persistence” as used herein is synonymous with the term “survival” and defines the length of time that a Treg or a population of Tregs can survive in a particular environment, e.g. ex vivo or in vivo (e.g. in a human patient or animal model). Typically, the Tregs of the present invention remain viable (i.e., as live cells) and can survive for longer periods of time than Tregs that have not been cultured in the presence of one or more GFLs (e.g. which are cultured under equivalent or the same conditions except for the absence of one or more GFLs). (“Not cultured in the presence of one or more GFLs” or “cultured in the absence of one or more GFLs” as used herein typically means that no GFLs are present in the culture medium. Particularly, GDNF is absent from the culture medium). As such, “increasing the persistence” or “enhancing the persistence” in accordance with the present invention means that the number of Tregs (particularly viable Tregs) is increased as compared to Tregs that have not been cultured in the presence of one or more GFLs (particularly than Tregs which are cultured under equivalent or the same conditions except for the absence of one or more GFLs) over the same period of time. A Treg as disclosed herein may have at least 10, 20, 30, 40, 50, 60, 70, 80 or 90% increased persistence or survival as compared to a Treg which has not been cultured in the presence of one or more GFLs. Persistence can be measured by for example, determining the amount or numbers of cells present at a particular time point (e.g. of Tregs cultured with and without one or more GFLs at a particular time point). The Tregs of the present invention may proliferate more than Tregs that have not been cultured in the presence of one or more GFLs (e.g. more than Tregs which are cultured under equivalent or the same conditions except for the absence of one or more GFLs). For example, the number of Tregs (and particularly the number of FOXP3+ Tregs) may be increased by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240% or 250% more than Tregs that have not been cultured in the presence of one or more GFLs,

Accordingly, the time it takes to expand the Tregs (e.g. to obtain or reach a particular number of Tregs) of the present invention is reduced compared to that of Tregs which have not been cultured in the presence of one or more GFLs (particularly than Tregs which are cultured under equivalent or the same conditions except for the absence of one or more GFLs). Therefore, in another aspect, the invention provides methods of reducing the expansion time of a Treg or a population of Tregs comprising culturing the Treg or population of Tregs with one or more GFLs, such as GDNF. Alternatively viewed, the invention provides use of one or more GFLs, such as GDNF, for reducing the expansion time of a Treg or population of Tregs.

The terms “expand” and “expansion” as defined herein refers to the induction of proliferation of a Treg or a Treg population of cells. The expansion of a population of cells may be measured for example by counting the number of cells present in a population. The phenotype of the cells may be determined by methods known in the art such as flow cytometry. A Treg or Treg population cultured in accordance with the invention typically has a reduced expansion time as compared to a Treg or Treg population which has not been cultured in the presence of one or more GFLs (e.g. under equivalent or the same conditions except for the absence of one or more GFLs). A reduced expansion time refers to a reduction in the time it takes to obtain a particular amount of cells (e.g. between about 15,000 to about 100,000 cells) when starting with a particular amount of cells (e.g. between about 3,000 to about 20,000 cells). The expansion time of a Treg or a population of Tregs in the methods of the present invention can be reduced by culturing the cells with one or more GFLs, wherein the expansion time is at least 10, 20, 30, 40, 50, 60, 70% less than for cells cultured in an equivalent medium without a GFL (e.g., cells which have not been contacted with a GFL). In particular, the expansion time is at least 50% less than for cells that have not been cultured with one or more GFLs. For example, a typical expansion time for a Treg or population of Tregs that has not been cultured with one or more GFLs is about 14 days. In the methods of the present invention, this expansion time may be reduced to 13, 12, 11, 10, 9, 8, 7, 6, 5, 4 or 3 days. In particular, the expansion time in the methods of the present invention may be reduced to 7 days. Alternatively viewed, the Treg or population of Tregs cultured in accordance with the methods or uses of the invention may have an increased expansion (e.g. an increased number of cells over a particular period of time as compared to a Treg or population of Tregs not cultured in the presence of one or more GFLs). Thus, one or more GFLs may be used to increase expansion of a Treg or a population of Tregs, e.g. by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240% or 250% as compared to a Treg or a population of Tregs not cultured in the presence of one or more GFLs.

The methods of the invention typically comprise a step of culturing. As defined herein, the terms “culture” or “culturing” mean to contact the Treg or population of Tregs with the one or more GFLs, e.g., by adding it to a cell culture medium that is known to be suitable for culturing Tregs, such as XVIVO™ media or TexMACS™ media.

Whilst not wishing to be bound by theory, it is postulated that the use of one or more GFLs, such as GDNF, functions to directly increase the number of Tregs in culture. Thus, in some aspects, GFLs may be seen as enhancing the persistence of Tregs by promoting their survival and increasing their proliferative capacity. This effect in turn reduces the expansion time required to achieve a therapeutic dose of Treg cells, which is another aspect of the invention. As defined herein, a “therapeutic dose” means a dose comprising a sufficient number of Tregs to produce a therapeutic effect in a patient, particularly at least 10×10⁶ Treg cells. For example, a therapeutic dose is at least 10×10⁶, 20×10⁶, 30×10⁶, 40×10⁶, 50×10⁶, 60×10⁶, 70×10⁶, 80×10⁶, 90×10⁶, 100×10⁶, 110×10⁶, 120×10⁶, 130×10⁶, 140×10⁶, 150×10⁶, 160×10⁶, 170×10⁶, 180×10⁶, 190×10⁶, 200×10⁶, 210×10⁶, 220×10⁶, 230×10⁶, 240×10⁶, 250×10⁶, 260×10⁶, 270×10⁶, 280×10⁶, 290×10⁶ or 300×10⁶. In a further example, a therapeutic dose may be from at least 10×10⁶ to 50×10⁶, 50×10⁶ to 100×10⁶, 100×10⁶ to 150×10⁶, 150×10⁶ to 200×10⁶, 200×10⁶ to 250 ×10⁶, 250×10⁶ to 300×10⁶ or 300×10⁶ to 350×10⁶. Preferably, a therapeutic dose is 200×10⁶.

Glial cell-lined derived neurotrophic factor ligands (GFLs) are neurotrophic factors that belong to the TGF-β superfamily, and include glial cell-line derived neurotrophic factor (GDNF), neurturin (NRTN), artemin (ARTN) and persephin (PSPN) which are highly homologous. The GFLs belong to the cystine-knot protein family, and they function as homodimers. GFLs are secreted proteins that are produced in the form of a precursor, preproGFL. The signal sequence is cleaved on secretion, and activation of the proGFL is thought to occur by proteolytic cleavage. All GFLs signal primarily through the same transmembrane receptor, tyrosine kinase Rearranged during transfection (RET), but GFLs can activate RET only in the presence of a co-receptor—GDNF Family Receptor a (GFRα). There are four GFRα receptors. In general, GDNF binds to GFRα1, NRTN binds to GFRα2, ARTN binds to GFRα3 and PSPN binds to GFRα4 but there is some crosstalk with GFLs binding to other GFRα receptors, e.g. GDNF can bind to GFRα2. Upon activation, RET is transphosphorylated and triggers intracellular signalling cascades (Airaksinen and Saarma, Nature Reviews Neuroscience, 2002, Volume 3:383-394).

Thus, the GFLs used in the present invention are typically selected from GDNF, NRTN, ARTN or PSPN. In a preferred embodiment, the GFL is GDNF. Furthermore, the GFLs used in the present invention may be selected from the prepro-protein forms (e.g., prepro-GDNF), the pro-protein forms (e.g. pro-GDNF), the mature protein forms (e.g., mature GDNF), N-terminally truncated forms (e.g., N-terminally truncated mature GDNF) or sequence variants, derivatives or fragments of any such proteins. In addition, human GDNF has five isoforms, human NRTN has one isoform, human ARTN has three isoforms and human PSPN has one isoform, all of which are encompassed for use by the present invention. The GFL may be a mammalian protein capable of having an effect on a corresponding mammalian cell, e.g. a human, mouse or rat protein. In a preferred embodiment, the GFLs used in the present invention are the human protein forms, preferably the mature human protein forms. In another embodiment, the GFLs used in the present invention are the mouse protein forms, preferably the mature mouse protein forms. The human protein forms may be limited to use on human cells. In another embodiment, the mouse or rat protein forms may be used, e.g. the mature mouse or rat protein forms. The mouse or rat protein forms may be limited to use on mouse or rat cells, respectively. Alternatively, the mouse or rat forms may be used on human cells.

In a further embodiment, the one or more GFLs is not used in combination with any other neurotrophic factors. In particular, the one or more GFLs is not used in combination with BDNF and CNTF or BDNF and IGF. More particularly, GDNF is not used in combination with BDNF and CNTF or BDNF and IGF.

Mature human GDNF, as used herein, typically comprises or consists of the 134 amino acids of native human GDNF, i.e., amino acids 78 to 211 of SEQ ID NO:5, and is processed into a dimer. Typically, the mature form of human GDNF comprises or consists of the amino acid sequence of SEQ ID NO:1. Typically, the prepro-protein form of human GDNF comprises or consists of the amino acid sequence of SEQ ID NO:5. Typically, the pro-protein form of human GDNF comprises or consists of the amino acid sequence of SEQ ID NO:9.

Mature human NRTN, as used herein, typically comprises or consists of the 102 amino acids of native human NRTN, i.e. amino acids 96 to 197 of SEQ ID NO:6, and is processed into a dimer. Typically, the mature form of human NRTN comprises or consists of the amino acid sequence of SEQ ID NO:2. Typically, the prepro-protein form of human NRTN comprises or consists of the amino acid sequence of SEQ ID NO:6. Typically, the pro-protein form of human NRTN comprises or consists of the amino acid sequence of SEQ ID NO:10.

Mature human ARTN, as used herein, typically comprises or consists of the 113 amino acids of native human ARTN, i.e. amino acids 108 to 220 of SEQ ID NO:7, and is processed into a dimer. Typically, the mature form of human ARTN comprises or consists of the amino acids of SEQ ID NO:3. Typically, the prepro-protein form of human ARTN comprises or consists of the amino acid sequence of SEQ ID NO:7. Typically, the pro-protein form of human ARTN comprises or consists of the amino acid sequence of SEQ ID NO:11.

Mature human PSPN, as used herein, typically comprises or consists of the 96 amino acids of native human PSPN, i.e. amino acids 61 to 156 of SEQ ID NO:8, and is processed into a dimer. Typically, the mature form of human PSPN comprises or consists of the amino acid sequence of SEQ ID NO:4. Typically, the prepro-protein form of human PSPN comprises or consists of the amino acid sequence of SEQ ID NO:8. Typically, the pro-protein form of human PSPN comprises or consists of the amino acid sequence of SEQ ID NO:12.

Mature mouse GDNF, as used herein, typically comprises or consists of the amino acid sequence of SEQ ID NO:13 and is processed into a dimer. Typically, the prepro-protein form of mouse GDNF comprises or consists of the amino acid sequence of SEQ ID NO:17. Typically, the pro-protein form of mouse GDNF comprises or consists of the amino acid sequence of SEQ ID NO:21.

Mature mouse NRTN, as used herein, typically comprises or consists of the amino acid sequence of SEQ ID NO:14 and is processed into a dimer. Typically, the prepro-protein form of mouse NRTN comprises or consists of the amino acid sequence of SEQ ID NO:18. Typically, the pro-protein form of mouse NRTN comprises or consists of the amino acid sequence of SEQ ID NO:22.

Mature mouse ARTN, as used herein, typically comprises or consists of the amino acid sequence of SEQ ID NO:15 and is processed into a dimer. Typically, the prepro-protein form of mouse ARTN comprises or consists of the amino acid sequence of SEQ ID NO:19. Typically, the pro-protein form of mouse ARTN comprises or consists of the amino acid sequence of SEQ ID NO:23.

Mature mouse PSPN, as used herein, typically comprises or consists of the amino acid sequence of SEQ ID NO:16 and is processed into a dimer. Typically, the prepro-protein form of mouse PSPN comprises or consists of the amino acid sequence of SEQ ID NO:20.

Mature rat GDNF, as used herein, typically comprises or consists of the amino acid sequence of SEQ ID NO:24 and is processed into a dimer. Typically, the prepro-protein form of rat GDNF comprises or consists of the amino acid sequence of SEQ ID NO:28.

Mature rat NRTN, as used herein, typically comprises or consists of the amino acid sequence of SEQ ID NO:25 and is processed into a dimer. Typically, the prepro-protein form of rat NRTN comprises or consists of the amino acid sequence of SEQ ID NO:29.

Mature rat ARTN, as used herein, typically comprises or consists of the amino acid sequence of SEQ ID NO:26 and is processed into a dimer. Typically, the prepro-protein form of rat ARTN comprises or consists of the amino acid sequence of SEQ ID NO:30. Typically, the pro-protein form of rat ARTN comprises or consists of the amino acid sequence of SEQ ID NO:32.

Mature rat PSPN, as used herein, typically comprises or consists of the amino acid sequence of SEQ ID NO:27 and is processed into a dimer. Typically, the prepro-protein form of rat PSPN comprises or consists of the amino acid sequence of SEQ ID NO:31.

Thus, in one embodiment GDNF comprises or consists of the sequence of SEQ ID NO:1, NRTN comprises or consists of the sequence of SEQ ID NO:2, ARTN comprises or consists of the sequence of SEQ ID NO:3, PSPN comprises or consists of the sequence of SEQ ID NO:4, or is a truncated form, mutated form or sequence variant thereof, wherein the sequence variant has about 95%, 90%, 85%, 80%, 75% or 70% sequence identity to SEQ ID NO:1, SEQ ID NO:2, SEQ ID NO:3 or SEQ ID NO:4.

In another embodiment, GDNF comprises or consists of the sequence of SEQ ID NO:5, NRTN comprises or consists of the sequence of SEQ ID NO:6, ARTN comprises or consists of the sequence of SEQ ID NO:7 and PSPN comprises or consists of the sequence of SEQ ID NO:8, or is a truncated form, mutated form or sequence variant thereof, wherein the sequence variant has about 95%, 90%, 85%, 80%, 75% or 70% sequence identity to SEQ ID NO:5, SEQ ID NO:6, SEQ ID NO:7 or SEQ ID NO:8.

In a further embodiment, GDNF comprises or consists of the sequence of SEQ ID NO:9, NRTN comprises or consists of the sequence of SEQ ID NO:10, ARTN comprises or consists of the sequence of SEQ ID NO:11 and PSPN comprises or consists of the sequence of SEQ ID NO:12, or is a truncated form, mutated form or sequence variant thereof, wherein the sequence variant has about 95%, 90%, 85%, 80%, 75% or 70% sequence identity to SEQ ID NO:9, SEQ ID NO:10, SEQ ID NO:11 or SEQ ID NO:12.

In another embodiment, GDNF comprises or consists of the sequence of SEQ ID NO:13, NRTN comprises or consists of the sequence of SEQ ID NO:14, ARTN comprises or consists of the sequence of SEQ ID NO:15 and PSPN comprises or consists of the sequence of SEQ ID NO:16, or is a truncated form, mutated form or sequence variant thereof, wherein the sequence variant has about 95%, 90%, 85%, 80%, 75% or 70% sequence identity to SEQ ID NO:13, SEQ ID NO:14, SEQ ID NO:15 or SEQ ID NO:16.

In another embodiment, GDNF comprises or consists of the sequence of SEQ ID NO:17, NRTN comprises or consists of the sequence of SEQ ID NO:18, ARTN comprises or consists of the sequence of SEQ ID NO:19 and PSPN comprises or consists of the sequence of SEQ ID NO:20, or is a truncated form, mutated form or sequence variant thereof, wherein the sequence variant has about 95%, 90%, 85%, 80%, 75% or 70% sequence identity to SEQ ID NO:17, SEQ ID NO:18, SEQ ID NO:19 or SEQ ID NO:20.

In another embodiment, GDNF comprises or consists of the sequence of SEQ ID NO:21, NRTN comprises or consists of the sequence of SEQ ID NO:22 and ARTN comprises or consists of the sequence of SEQ ID NO:23, or is a truncated form, mutated form or sequence variant thereof, wherein the sequence variant has about 95%, 90%, 85%, 80%, 75% or 70% sequence identity to SEQ ID NO:21, SEQ ID NO:22 or SEQ ID NO:23.

In further embodiment, GDNF comprises or consists of the sequence of SEQ ID NO:24, NRTN comprises or consists of the sequence of SEQ ID NO:25, ARTN comprises or consists of the sequence of SEQ ID NO:26 and PSPN comprises or consists of the sequence of SEQ ID NO:27, or is a truncated form, mutated form or sequence variant thereof, wherein the sequence variant has about 95%, 90%, 85%, 80%, 75% or 70% sequence identity to SEQ ID NO:24, SEQ ID NO:25, SEQ ID NO:26 or SEQ ID NO:27.

In further embodiment, GDNF comprises or consists of the sequence of SEQ ID NO:28, NRTN comprises or consists of the sequence of SEQ ID NO:29, ARTN comprises or consists of the sequence of SEQ ID NO:30 and PSPN comprises or consists of the sequence of SEQ ID NO:31, or is a truncated form, mutated form or sequence variant thereof, wherein the sequence variant has about 95%, 90%, 85%, 80%, 75% or 70% sequence identity to SEQ ID NO:28, SEQ ID NO:29, SEQ ID NO:30 or SEQ ID NO:31.

In another embodiment, ARTN comprises or consists of the sequence of SEQ ID NO:34 or is a truncated form, mutated form or sequence variant thereof, wherein the sequence variant has about 95%, 90%, 85%, 80%, 75% or 70% sequence identity to SEQ ID NO:34.

In a preferred embodiment, GDNF comprises or consists of the sequence of SEQ ID NO:1 or SEQ ID NO:13, or is a truncated form, mutated form or sequence variant thereof, wherein the sequence variant has about 95%, 90%, 85%, 80%, 75% or 70% sequence identity to SEQ ID NO:1 or 13.

In a preferred embodiment, NRTN comprises or consists of the sequence of SEQ ID NO:2 or SEQ ID NO:14, or is a truncated form, mutated form or sequence variant thereof, wherein the sequence variant has about 95%, 90%, 85%, 80%, 75% or 70% sequence identity to SEQ ID NO:2 or 14.

In a preferred embodiment, ARTN comprises or consists of the sequence of SEQ ID NO:3 or SEQ ID NO:15, or is a truncated form, mutated form or sequence variant thereof, wherein the sequence variant has about 95%, 90%, 85%, 80%, 75% or 70% sequence identity to SEQ ID NO:3 or 15.

In a preferred embodiment, PSPN comprises or consists of the sequence of SEQ ID NO:4 or SEQ ID NO:16, or is a truncated form, mutated form or sequence variant thereof, wherein the sequence variant has about 95%, 90%, 85%, 80%, 75% or 70% sequence identity to SEQ ID NO:4 or 16.

It is contemplated that the sequence of GFLs can be changed without changing or substantially changing the biological activity of the growth factors. Thus, in addition to the specific amino acid sequences mentioned herein, also encompassed is the use of variants, or derivatives and fragments thereof.

The term “derivative” or “variant” as used interchangeably herein, in relation to proteins or polypeptides of the present invention includes any substitution of, variation of, modification of, replacement of, deletion of and/or addition of one (or more) amino acid residues from or to the sequence providing that the resultant protein or polypeptide retains the desired function. For example, where the derivative or variant is a derivative or variant of a GFL, the desired function may be the ability of the derivative or variant to increase the persistence of a Treg or a population of Tregs, where the derivative or variant is a signalling domain, the desired function may be the ability of that domain to signal (e.g. activate or inactivate a downstream molecule), where the derivative or variant is a transcription factor (e.g. FOXP3), the desired function may be the ability of the transcription factor to bind to target DNA and/or to induce transcription or where the derivative or variant is a safety switch polypeptide, the desired function may be the ability of that polypeptide to induce cell death e.g. upon binding of a molecule thereto. Alternatively viewed, the variants or derivatives referred to herein are functional variants or derivatives. For example, the variant or derivative may have at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding reference sequence or may have an increased level of function (e.g. increased by at least 10%, at least 20%, at least 30%, at least 40% or at least 50%). For example, a variant GFL of the invention may have at least 10, 20, 30, 40, 50, 60, 70, 80 or 90% of the function of the reference GFL protein or polypeptide.

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 1 below.

TABLE 1

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

The derivative may be a homologue. The term “homologue” as used herein means an entity having a certain homology with the wild-type amino acid sequence and the wild-type nucleotide sequence. The term “homology” can be equated with “identity”.

A homologous or variant sequence may include an amino acid sequence which may be at least 70%, 75%, 85% or 90% identical, preferably at least 95%, 96%, 97%, 98% or 99% identical to the subject sequence. Typically, the variants will comprise the same active sites etc. as the subject amino acid sequence. Although homology can also be considered in terms of similarity (i.e. amino acid residues having similar chemical properties/functions), in the context herein it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence. Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally, e.g. is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

While the quantity and frequency of administration of the one or more GFLs to the Tregs in culture will be determined by such factors as the condition of the cells and the time in culture, the inventors have shown that culturing Tregs with a single dose of a GFL advantageously enhances their persistence and proliferation in culture. Thus, in some embodiments, the cells are cultured with a single, one-off dose of the one or more GFLs. The one or more GFLs may be administered at a concentration of about 1 to 40 ng/ml. For example, the one or more GFLs may be administered at a concentration of about 1, 1.25, 1.5, 1.75, 2, 2.5, 3, 4, 5, 6, 7, 8, 9, 10, 11, 12, 13, 14, 15, 16, 17, 18, 19, 20, 22.5, 25, 27.5, 30, 32.5, 35, 37.5 or 40 ng/ml. In another example, the one or more GFLs may be administered at a concentration of about 2.5-30, 5-20, 7.5-15, 8-14 or 10-12 ng/ml. In one embodiment, the GFL may be administered at a concentration of 10 ng/ml. Alternatively, the one or more GFLs may be administered in multiple doses, for example, if the Treg or Treg population of the invention is in culture for 6 days, the one or more GFLs may be administered in a single dose on day 1 and then again on day 4 of the culture.

The inventors have also shown that the persistence/survival of the Treg or Treg population is increased under both resting and during stimulation conditions. An optimal dose of the one or more GFLs (such as GDNF) for increasing Treg persistence at rest may be from about 1, 1.25, 1.5, 2, 2.5, 3, 3.5, 4, 4.5, 5, 6, 7, 8, 9, 10, 15, 20, 25, 30, 35 or 40 ng/ml. For example, the one or more GFLs may be administered to a resting cell at a concentration of about 1-10, 1-5, 1-3, 2-5, 2-4 or 2-3 ng/ml. An optimal dose of the one or more GFLs (such as GDNF) for increasing Treg persistence during stimulation may be from about 1, 1.25, 1.5, 2, 2.5, 3, 3.5, 4, 4.5, 5, 6, 7, 8, 9, 10, 15, 20, 25, 30, 35 or 40 ng/ml. For example, the one or more GFLs may be administered to a stimulated cell at a concentration of about 1-20, 1-10, 2-18, 3-15, 5-15, 7.5-12.5, 5-10, 8-11, or 9-10 ng/ml. Preferably, an optimal dose of the one or more GFLs (such as GDNF) for increasing Treg persistence at rest may be about 2.5 ng/ml, whereas an optimal dose for increasing Treg persistence during stimulation may be about 10 ng/ml. Resting conditions as defined herein mean that the cells do not experience any antigen stimulation (e.g. via their TCR). For example, they may be rested by culturing in a suitable Treg media supplemented with IL-2 but which is absent of anything that could stimulate the cells (e.g. anti-CD3 or anti-CD28 beads). Thus, under resting conditions the Treg cells are functionally quiescent. Stimulation conditions as defined herein mean that the cells are activated by antigen stimulation (e.g. via their TCR). For example, they may be stimulated by culturing in a suitable Treg media supplemented with IL-2 and anti-CD3/anti-CD28 beads, which activate the cells via their TCR. Thus, under stimulation conditions the Treg cells are functionally active.

As described herein, “one or more GFLs” can be used in the methods, culture media, combination therapies or products, and kits of the present invention, i.e. one GFL may be used alone or a combination of GFLs may be used, e.g. two GFLs, three GFLs, four GFLs or more. Where a combination of GFLs is used, the combination may particularly comprise GDNF with one or with multiple GFLs. For example, the combination may be GDNF and NRTN, or GDNF, NRTN and ARTN or, GDNF, NRTN, ARTN and PSPN. However, any combination of GFLs may be used in accordance with the present invention. In a particular embodiment, where one GFL is used alone, the GFL may be GDNF.

As also described herein, the one or more GFLs may not be used in combination with any other neurotrophic factors. In particular, the one or more GFLs may not be used in combination with BDNF and CNTF or BDNF and IGF. Specifically, GDNF may not be used in combination with BDNF and CNTF or BDNF and IGF.

“Regulatory T cells (Tregs)” or “T regulatory cells” are immune cells with immunosuppressive function that control cytopathic immune responses and are essential for the maintenance of immunological tolerance. As used herein, the term Treg refers to a T cell with immunosuppressive function.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs. Antigen-specific Tconv cells co-cultured with Tregs as described herein may proliferate 5%, 10%, 20%, 30%, 40%, 50%, 60%, 70%, 90%, 95% or 99% less than the same Tconv cells cultured in the absence of Tregs as described herein.

Antigen-specific Tconv cells co-cultured with Tregs may express at least 10%, at least 20%, at least 30%, at least 40%, at least 50%, or at least 60% less effector cytokine than corresponding Tconv cells cultured in the absence of Tregs. The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Tregs may also express CTLA-4 (cytotoxic T-lymphocyte associated molecule-4) or GITR (glucocorticoid-induced TNF receptor).

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

A Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Different subpopulations of Tregs are known to exist, including naïve Tregs (CD45RA⁺FoxP3^{low}), effector/memory Tregs (CD45RA⁻FoxP3^{high}) and cytokine-producing Tregs (CD45RA⁻FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc., expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺.

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out as part of the invention, and methods for determining the presence and/or levels of expression of cell markers are well-known in the art and include, for example, flow cytometry, using commercially available antibodies.

Thus, the methods and uses of the invention relate to a Treg or a plurality of Tregs, e.g., a population of Tregs. Also encompassed is the use of a medicament comprising a plurality of Tregs as defined above, e.g. a population of Tregs. Thus, in some embodiments, the medicament comprises a population of Tregs. It will be appreciated that not all cells within a cell population may express the Treg markers described above to the same extent. Thus, a population of Tregs may comprise distinct and identifiable sub-populations of Tregs as defined above. For utility in the methods and uses of the invention, it may be sufficient that at least about 50% of the cells in the population are identifiable as Tregs, preferably at least about 60, 70, 80, 90 or 95% of the population are identifiable as Tregs, preferably naïve Tregs.

The Tregs for use in the invention may be modified or engineered Tregs. An “engineered cell” or “engineered Treg” as used herein means a cell which has been modified to comprise or express a polynucleotide that is not naturally encoded by the cell. It will be appreciated that Tregs may be engineered to express numerous polynucleotides to facilitate their use in the invention. For instance, the Treg may be engineered to express a chimeric receptor, e.g. a chimeric antigen receptor (CAR) or an exogenous T cell receptor (TCR), that enhances the utility of the Treg in one or more uses described herein, e.g. that makes the engineered Treg more effective at treating and/or preventing a disorder as described herein than a corresponding Treg not expressing the chimeric receptor. Additionally or alternatively, the Treg may be engineered to express an polypeptide that improves other properties of the Treg, e.g. a polypeptide that improves the cryopreservation of the cell, functions to maintain the identity of the cell, functions to improve the proliferation of the cell and/or in vivo persistence, functions to induce cell death in response to stimulus, e.g. a safety switch polypeptide etc. For instance, the engineered Treg may comprise an exogenous polynucleotide encoding FOXP3 or an IL2 receptor polypeptide or portion thereof. In a representative example, the engineered Treg may be a Treg as described in any one of WO 2020/044055, WO2021/170666, WO 2021/239812, WO 2019/202323, WO 2021/079149, WO 2022/043483 (all of which are herein incorporated by reference) or may be engineered as described in any of the aforementioned applications or a combination thereof. Thus, in some embodiments, the Treg or population of Tregs is an engineered Treg or population of Tregs, wherein the Treg or population of Tregs is engineered to express a CAR, an exogenous TCR and/or an exogenous FOXP3 polypeptide. In other words, the Treg is a CAR-Treg, i.e., a Treg engineered to express a CAR or a TCR, or a CAR/TCR and FOXP3.

The methods and uses of the present invention may therefore relate to a cell population comprising an engineered Treg cell (a plurality of engineered Treg cells). Accordingly, the medicament of the present invention may comprise a cell population comprising an engineered Treg cell (a plurality of engineered Treg cells). The cell population may have been transduced with a vector encoding a polynucleotide that is not naturally encoded by the cell. A proportion of the cells of the cell population may express the polynucleotide. Thus, where the engineered Treg comprises a polynucleotide encoding a chimeric receptor, e.g. a CAR, a proportion of the cells of the cell population may express the chimeric receptor, e.g. CAR, at the cell surface. Furthermore, a proportion of the cells of the cell population may co-express a chimeric receptor, e.g. CAR, and a further polypeptide (e.g. an accessory protein) as described above, e.g. exogenous FOXP3, safety switch etc. Thus, it will be appreciated that not all cells within a cell population may express the polynucleotide(s) that is (are) not naturally encoded by the cell, e.g. chimeric receptor. Particularly, at least 50, 60, 70, 80, 90, 95 or 99% of cells express the polynucleotide(s) that is (are) not naturally encoded by the cell, e.g. chimeric receptor.

The term “chimeric receptor” refers to a receptor protein comprising linked domains from two or more proteins, e.g. an exodomain from a first protein and an endodomain from a second protein. Typically, at least one of the domains is derived from a receptor protein. Thus, a chimeric receptor may be viewed as an “engineered receptor” and these terms are used interchangeably herein.

A chimeric receptor may comprise linked domains on a single polypeptide chain (a single contiguous chain) or may comprise two or more polypeptide chains (a multichain chimeric receptor), wherein at least one of the polypeptide chains comprises linked domains from two or more proteins. Thus, a chimeric receptor may comprise at least two polypeptide chains which may associate with each other when co-expressed, particularly through their transmembrane domains, and/or through an alternative dimerization site. Typically, each polypeptide chain within a multichain chimeric receptor will comprise of two or more linked domains, for example, a first polypeptide chain may comprise an extracellular domain and a transmembrane domain, and a second polypeptide may comprise a transmembrane domain and an endodomain, or a first polypeptide may comprise an extracellular domain, a transmembrane domain and an endodomain and a second polypeptide may comprise a transmembrane domain and an endodomain. However, it is also possible for one of the polypeptide chains to only comprise a single domain, typically an endodomain. It will therefore be appreciated that a chimeric receptor for use in the invention may comprise more than one of a particular domain within the same or within different polypeptide chains. For example, where the chimeric receptor is a multichain chimeric receptor, the chimeric receptor may comprise two transmembrane domains and/or two endodomains which may be the same or different.

A “Chimeric antigen receptor”, “CAR” or “CAR construct” refers to engineered receptors which can confer an antigen specificity onto cells (e.g. immune cells, such as Tregs). As discussed above, a CAR may comprise a single polypeptide chain or may comprise two or more polypeptide chains (e.g. a first polypeptide chain and a second polypeptide chain). In particular, a CAR enables a cell to bind specifically to a particular antigen, e.g. a target molecule such as a target protein, whereupon a signal is generated by the endodomain (comprising an intracellular signalling domain) of the CAR, e.g. a signal resulting in activation of the cell. CARs are also known as artificial T-cell receptors, chimeric T-cell receptors or chimeric immunoreceptors. Thus, the chimeric receptor for use in the invention may function to confer Tregs expressing the receptor with the ability to bind specifically to ligands associated with the conditions and disorders to be treated according to the invention.

The structure of CARs is well-known in the art and several generations of CARs have been produced. For instance, as a minimum a CAR may contain an extracellular antigen-specific targeting region, antigen binding domain or ligand binding domain, which is or forms part of the exodomain (also known as the extracellular domain or ectodomain) of the CAR, a transmembrane domain, and an intracellular signalling domain (which is or is comprised within an endodomain). However, the CAR may contain further domains to improve its functionality, e.g. one or more co-stimulatory domains to improve T cell proliferation, cytokine secretion, resistance to apoptosis, and in vivo persistence. As discussed above, the CAR may comprise more than one polypeptide chain and thus the domains may occur within the same or within different polypeptides, typically which associate with one another. Thus, a CAR may comprise two polypeptides wherein the first polypeptide comprises the extracellular domain, a transmembrane domain and optionally an endodomain, and the second polypeptide comprises an endodomain and optionally a transmembrane domain. Particularly, at least one endodomain in a multichain CAR will comprise an intracellular signalling domain.

Thus, a CAR construct generally comprises an antigen or ligand binding domain, optionally a hinge domain, which functions as a spacer to extend the antigen or ligand binding domain away from the plasma membrane of the cell (e.g. immune cell, e.g. Treg) on which it is expressed, a transmembrane domain, an intracellular signalling domain (e.g. the signalling domain from the zeta chain of the CD3 molecule (CD3ζ) of the TcR complex, or an equivalent) and optionally one or more co-stimulatory domains, which may assist in signalling or functionality of the cell expressing the CAR. A CAR may also comprise a signal or leader sequence or domain which functions to target the protein to the membrane and may form part of the exodomain of the CAR. The different domains may be linked directly or by linkers, and/or may occur within different polypeptides, e.g. within two polypeptides which associate with one another.

As discussed herein, some of the methods of the invention find particular utility in the immunomodulation of a subject. In certain aspects, the immunomodulation is for treating and/or preventing rejection of a transplant, particularly in liver transplant recipients. In this respect, antigens associated with organ transplants and/or cells associated with transplanted organs include a HLA antigen present in the transplanted organ but not in the patient, an organ-specific, tissue-specific or cell-specific antigen (e.g. a liver-specific antigen) or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11. Thus, in some embodiments, the engineered Treg for use in the invention comprises a chimeric receptor (e.g. a CAR) that selectively binds to a HLA antigen, e.g. HLA-A2, present in the transplanted organ but not in the patient, an organ-specific antigen (e.g. a liver-specific antigen such as Na+/taurocholate co-transporting polypeptide (NTCP)), a tissue-specific antigen, a cell-specific antigen, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11. In a representative example, the engineered Treg for use in the invention comprises a HLA CAR as described in WO 2018/001874, WO 2020/201230 or WO 2022/043483 (which are all herein incorporated by reference).

As mentioned above, the cell or cell population of the invention may further comprise additional polypeptides, particularly exogenous polypeptides, such as a FOXP3 and/or safety switch polypeptide. The polypeptides of the present invention, e.g., the CAR, FOXP3 and safety switch, may be encoded by a single nucleic acid molecule. The nucleic acid molecule may comprise nucleotide sequences encoding self-cleavage sequences in between the encoded polypeptides, allowing the polypeptides to be expressed and/or produced as separate, or discrete components. By this it is meant that, although the polypeptides are encoded by a single nucleic acid molecule, through “cleavage” during or after translation at the encoded cleavage sites, they may be expressed or produced as separate polypeptides, and thus at the end of the protein production process in the cell, they may be present in the cell as separate entities, or separate polypeptide chains. Alternatively, additional exogenous polypeptides may be encoded by separate nucleic acid molecules or vectors.

By “discrete” or “separate” polypeptides it is meant that the polypeptides are not linked to one another and are physically distinct. Indeed, following expression, they are located in different, or separate cellular locations. The CAR, FOXP3 and safety switch polypeptide are thus ultimately expressed as single and separate components. The CAR is expressed as a cell surface molecule. The safety switch polypeptide may be expressed inside a cell, or on the cell surface. In a particular embodiment, the safety switch polypeptide and the CAR are expressed on the surface of a cell which is intended for ACT. The FOXP3 is expressed inside the cell, where it can exert its effect as a transcription factor to regulate cell development and/or activity, as described further below.

The safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, in this case the CAR, which when expressed allows the cell to be deleted to turn off expression of the transgene (CAR). A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9. However, it is preferred for the suicide moiety to be, or to comprise, an epitope which is recognised by a cell-deleting antibody or other binding molecule capable of eliciting deletion of the cell. In such an embodiment, the safety switch polypeptide is expressed on the surface of a cell.

The term “delete” as used herein in the context of cell deletion is synonymous with “remove” or “ablate” or “eliminate”. The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. Biosimilars for Rituximab are available and may be used. A person of skill in the art is readily able to use routine methods to prepare an antibody having the binding specificity of Rituximab using the available amino acid sequences therefor.

CAR-Tregs that also express a safety switch polypeptide comprising this sequence can be selectively killed using the antibody Rituximab, or an antibody having the binding specificity of Rituximab. The safety switch polypeptide is expressed on the cell surface and when the expressed polypeptide is exposed to or contacted with Rituximab, or an antibody with the same binding specificity, death of the cell ensues.

Thus, Rituximab, or an antibody having the binding specificity thereof, may be provided for use in ACT in combination with a Treg cell of the invention. The Treg cell and the Rituximab or equivalent antibody may be provided in a kit, or as a combination product.

For example, the suicide constructs of WO2013/153391 or WO2021/239812 (both incorporated herein by reference) may be used in a cell or cell population (e.g., Treg or Treg population) as described herein.

The nucleic acid molecule of the present invention may be designed to increase FOXP3 expression in cells (e.g., Tregs) by introducing into the cells a nucleotide sequence encoding FOXP3, which term is synonymous with the term “a FOXP3 polypeptide” or “exogenous FOXP3 polypeptide”. The nucleic acid molecule may be introduced into cells via constructs and vectors that contain it. The nucleic acid molecule, and constructs and vectors containing it, thus provide a means for increasing FOXP3 in a cell, e.g., in a Treg or a CD4+ cell.

“FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

“Increasing FOXP3 expression” means to increase the levels of FOXP3 mRNA and/or protein in a Treg cell (or population of Treg cells) in comparison to a corresponding Treg cell which has not been modified (or population of Treg cells) by introduction of the nucleic acid molecule, construct or vector. For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells).

Suitably, the level of FOXP3 mRNA and/or protein in a modified Treg cell (or a population of such Treg cells) may be increased to at least 1.5-fold greater, 2-fold greater, or 5-fold greater than the level in a corresponding Treg cell which has not been so modified (or population of such cells).

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e., a polypeptide able to bind FOXP3 target DNA and function as a transcription factor regulating development and function of Tregs. Particularly, a FOXP3 polypeptide may have the same or similar activity to wildtype FOXP3 (SEQ ID NO:33), e.g., may have at least 40, 50, 60, 70, 80, 90, 95, 100, 110, 120, 130, 140 or 150% of the activity of the wildtype FOXP3 polypeptide. Thus, a FOXP3 polypeptide encoded by the nucleotide sequence in the nucleic acid, construct or vector described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24:209-26, incorporated herein by reference). As discussed in detail below, FOXP3 or a FOXP3 polypeptide includes functional fragments, variants, and isoforms thereof, e.g., of SEQ ID NO:33.

A “functional fragment of FOXP3” may refer to a portion or region of a FOXP3 polypeptide or a polynucleotide (i.e., nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp.665-675; Lopes, J. E., et al., 2006. The Journal of Immunology, 177(5), pp.3133-3142; and Lozano, T., et al, 2013. Frontiers in oncology, 3, p.294. Further, a N and C terminally truncated FOXP3 fragment is described within WO2019/241549 (incorporated herein by reference), for example, having the sequence SEQ ID NO:61 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide, e.g., to SEQ ID NO:33. FOXP3 variants may have the same or similar activity to a wildtype FOXP3 polypeptide or polynucleotide, e.g., may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g., at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO:33, for example S418E and/or S422A, as described in WO2019/241549 (incorporated herein by reference) and are set out in SEQ ID NOs:34 to 36, which represent the aa418, aa422 and aa418 and aa422 mutants respectively.

Suitably, the FOXP3 polypeptide encoded by a nucleic acid molecule, construct or vector as described herein may comprise or consist of the polypeptide sequence of a human FOXP3, such as UniProtKB accession Q9BZS1 (SEQ ID NO:33), or a functional fragment or variant thereof.

In some embodiments of the invention, the FOXP3 polypeptide comprises or consists of an amino acid sequence which is at least 70% identical to SEQ ID NO:33 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO:33 or a functional fragment thereof. In some embodiments, the FOXP3 polypeptide comprises or consists of SEQ ID NO:33 or a functional fragment thereof.

In some embodiments, as discussed above, the FOXP3 polypeptide may comprise mutations at residues 418 and/or 422 of SEQ ID NO:33, as set out in SEQ ID NO:34, SEQ ID NO:35, or SEQ ID NO:36.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of SEQ ID NO:37 or a functional variant thereof having at least 80, 85, 90, 95 or 99% identity thereto.

Suitably, the FOXP3 polypeptide may be a variant of SEQ ID NO:33, for example a natural variant. Suitably, the FOXP3 polypeptide is an isoform of SEQ ID NO:33. For example, the FOXP3 polypeptide may comprise a deletion of amino acid positions 72-106 relative to SEQ ID NO:33. Alternatively, the FOXP3 polypeptide may comprise a deletion of amino acid positions 246-272 relative to SEQ ID NO:33.

Suitably, the FOXP3 polypeptide comprises SEQ ID NO:38 or a functional fragment thereof. SEQ ID NO:38 represents an Illustrative FOXP3 polypeptide.

Suitably the FOXP3 polypeptide comprises or consists of an amino acid sequence which is at least 70% identical to SEQ ID NO:38 or a functional fragment thereof. Suitably, the FOXP3 polypeptide comprises an amino acid sequence which is at least 80%, at least 85%, at least 90%, at least 95%, at least 98% or at least 99% identical to SEQ ID NO:38 or a functional fragment thereof. In some embodiments, the FOXP3 polypeptide comprises or consists of SEQ ID NO:38 or a functional fragment thereof.

Suitably, the FOXP3 polypeptide may be a variant of SEQ ID NO:38, for example a natural variant. Suitably, the FOXP3 polypeptide is an isoform of SEQ ID NO:38 or a functional fragment thereof. For example, the FOXP3 polypeptide may comprise a deletion of amino acid positions 72-106 relative to SEQ ID NO:38. Alternatively, the FOXP3polypeptide may comprise a deletion of amino acid positions 246-272 relative to SEQ ID NO:38.

Suitably, the polynucleotide encoding a FOXP3 polypeptide comprises or consists of a nucleotide sequence set forth in SEQ ID NO:39, which represents an illustrative FOXP3 nucleotide sequence.

In some embodiments of the invention, the polynucleotide encoding the FOXP3 polypeptide or variant comprises nucleotide sequence which is at least 70% identical to SEQ ID NO:39 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO:39 or a fragment thereof which encodes a functional FOXP3 polypeptide. In some embodiments of the invention, the polynucleotide encoding the FOXP3 polypeptide or variant comprises or consists of SEQ ID NO:39 or a fragment thereof which encodes a functional FOXP3 polypeptide.

Suitably, the polynucleotide encoding a FOXP3 polypeptide comprises or consists of a polynucleotide sequence set forth in SEQ ID NO:40, which represents another illustrative FOXP3 nucleotide.

In some embodiments of the invention, the polynucleotide encoding the FOXP3 polypeptide or variant comprises a nucleotide sequence which is at least 70% identical to SEQ ID NO:40 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO:40 or a fragment thereof which encodes a functional FOXP3 polypeptide. In some embodiments of the invention, the polynucleotide encoding the FOXP3 polypeptide or variant comprises or consists of SEQ ID NO:40 or a fragment thereof which encodes a functional FOXP3 polypeptide.

A skilled person will appreciate that FOXP3 expression within a Treg may be increased indirectly by introducing a polynucleotide into the cell which encodes a protein which increases transcription and/or translation of FOXP3 or which increases the half-life (e.g., by at least 10, 20, 30, 40, 50, 60, 70, 80 or 90%) or function of FOXP3 (e.g. determined by suppressive ability of a transduced Treg, measured as previously discussed). For example, it may be possible to introduce a polynucleotide into a Treg which increases transcription of endogenous FOXP3 by interacting with the endogenous FOXP3 promoter or non-coding sequences (CNS, e.g., CNS1, 2 or 3) which are found upstream of the coding region.

Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

Methods for engineering cells are known in the art and include, but are not limited to, genetic modification of cells, e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid molecule into a Treg cell. Non-viral technologies such as amphipathic cell penetrating peptides may be used to introduce a nucleic acid molecule into a Treg cell for use in the present invention.

Accordingly, an engineered cell (i.e. Treg) is a cell which has been modified or whose genome has been modified e.g. by transduction or by transfection. Suitably, an engineered cell is a cell that has been modified or whose genome has been modified by retroviral transduction. Suitably, an engineered cell is a cell which has been modified or whose genome has been modified by lentiviral transduction.

As used herein, the term “introduced” refers to methods for inserting foreign DNA or RNA into a cell and includes both transduction and transfection methods. Transfection is the process of introducing nucleic acids into a cell by non-viral methods. Transduction is the process of introducing foreign DNA or RNA into a cell via a viral vector. Engineered Treg cells according to the present invention may be generated by introducing DNA or RNA, e.g.

encoding a polypeptide (e.g. chimeric receptor), by one of many means including transduction with a viral vector, transfection with DNA or RNA. Cells may be activated and/or expanded prior to, or after, the introduction of a polynucleotide, for example by treatment with an anti-CD3 monoclonal antibody or both anti-CD3 and anti-CD28 monoclonal antibodies. Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

Engineered Tregs of the present invention may be made by: introducing to a cell (e.g. by transduction or transfection) the nucleic acid molecule/polynucleotide, construct or vector as defined herein.

Suitable cells are discussed further below, but the cell may be from a sample isolated from a subject. The subject may be a donor subject, or a subject for therapy (i.e., the cell may be an autologous cell, or a donor cell, for introduction to another recipient, e.g., an allogeneic cell).

The cell may be generated by a method comprising the following steps:
(i) isolation of a cell-containing sample from a subject or provision of a cell-containing sample; and
(ii) introduction into (e.g., by transduction or transfection) the cell-containing sample of a nucleic acid molecule, construct, or vector as defined herein, to provide a population of engineered cells.

The cell-containing sample may be cultured with one or more GFLs prior to and/or after step (ii) of the above method. In particular, the GFL may be GDNF. Similarly, a Treg-enriched sample may be isolated from, enriched, and/or generated from the cell-containing sample prior to and/or after step (ii) of the above method. For example, isolation, enrichment, generation of Tregs and/or culturing with one or more GFLs may be performed prior to and/or after step (ii) to isolate, enrich or generate a Treg-enriched sample in accordance with the present invention. Isolation and/or enrichment from a cell-containing sample may be performed after step (ii) to enrich for cells and/or Tregs comprising the CAR, the nucleic acid molecule/polynucleotide, the construct and/or the vector as described herein. The cell-containing sample of step (i) may be obtained from a subject, e.g. directly from peripheral blood or via leukapheresis, or from a cell source, such as from iPSC cells, to provide iPSC derived Tregs.

Therefore, in one embodiment, the cell may be generated by a method comprising the following steps:
(i) isolation of a cell-containing sample from a subject or provision of a cell-containing sample;
(ii) enrichment of the cell-containing sample to generate a Treg-enriched sample;
(iii) introduction into (e.g., by transduction or transfection) the Treg-enriched sample of a nucleic acid molecule, construct, or vector as defined herein, to provide a population of engineered cells;
(iv) expansion of the Treg-enriched sample; and
(v) culture of the expanded Treg-enriched sample with one or more GFLs.

The one or more GFLs may be GDNF. Steps (iv) and (v) of the above method may be combined so that the cells are cultured with one or more GFLs during the expansion process. In addition, the culture step, step (v), of the above method may include culture of the expanded Treg-enriched sample with anti-CD3/anti-CD28 antibodies/beads and/or IL-2, as well as the one or more GFLs.

Alternatively, in another embodiment, the cell may be generated by a method comprising the following steps:
(i) isolation of a cell-containing sample from a subject or provision of a cell-containing sample;
(ii) enrichment of the cell-containing sample to generate a Treg-enriched sample;
(iii) culture of the Treg-enriched sample with one or more GFLs;
(vi) introduction into (e.g., by transduction or transfection) the Treg-enriched sample of a nucleic acid molecule, construct, or vector as defined herein, to provide a population of engineered cells; and
(vii) expansion of the Treg-enriched sample.

The one or more GFLs may be GDNF. Steps (iii) and (vii) of the above method may be combined so that the cells are cultured with one or more GFLs during the expansion process. In addition, the culture step, step (iii), of the above method may include culture of the Treg-enriched sample with anti-CD3/anti-CD28 antibodies/beads and/or IL-2, as well as the one or more GFLs.

The cell of the present invention may not be genetically engineered. Therefore, in any of the above methods, the transduction or transfection step may be removed. For example, in one embodiment, the cell may be generated by a method comprising the following steps:
(i) isolation of a cell-containing sample from a subject or provision of a cell-containing sample;
(ii) enrichment of the cell-containing sample to generate a Treg-enriched sample;
(iii) expansion of the Treg-enriched sample; and
(iv) culture of the expanded Treg-enriched sample with one or more GFLs.

The one or more GFLs may be GDNF. In one embodiment, steps (iii) and (iv) of the above method are combined so that the cells are cultured with one or more GFLs during the expansion process. In addition, the culture step, step (iv), of the above method may include culture of the expanded Treg-enriched sample with anti-CD3/anti-CD28 antibodies/beads and/or IL-2, as well as the one or more GFLs.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example, from Tcon cells by introducing DNA or RNA coding for FOXP3 and/or from ex-vivo differentiation of inducible progenitor cells or embryonic progenitor cells. Methods for isolating and/or enriching Treg cells are also known in the art.

Similarly, an Treg-enriched sample may be expanded by any method known to those of skill in the art and discussed above, for example, by stimulating the cells with anti-CD3/anti-CD28 antibodies/beads.

Suitably, an engineered Treg cell may be generated by a method comprising the following steps:
(i) isolation of a target-cell enriched sample from a subject or provision of a target cell-enriched sample; and
(ii) introduction into (e.g., by transduction or transfection) the target cell-enriched sample of a nucleic acid, construct or vector as defined herein, to provide a population of engineered Treg cells. The target cell may be a Treg cell, or precursor or a progenitor thereof.

The invention also encompasses a Treg or population of Tregs obtainable or obtained by a method of the invention, e.g. by a method of culturing said Treg or population of Tregs with one or more GFLs, e.g. GDNF.

In another aspect, the invention provides a method of immunomodulation of a subject in need thereof comprising the steps of:
(a) culturing a regulatory T cell (Treg) or a population of Tregs ex vivo with one or more glial cell line-derived neurotrophic factor (GDNF) family ligands (GFLs);
(b) formulating the Treg or population of Tregs into a medicament; and
(c) administering the medicament to the subject.

Any of the above method steps of generating a Treg cell may be used prior to step (a) of the method of immunomodulation. For example, in one embodiment, a method of immunomodulation of a subject in need thereof may comprise the steps of:
(a) isolation of a cell-containing sample from a subject or provision of a cell-containing sample;
(b) enrichment of the cell-containing sample to generate a Treg-enriched sample;
(c) introduction into (e.g., by transduction or transfection) the Treg-enriched sample of a nucleic acid molecule, construct, or vector as defined herein, to provide a population of engineered cells;
(d) expansion of the population of engineered cells;
(e) culture of the expanded population of engineered cells ex vivo with one or more GFLs;
(f) formulation of the expanded population of engineered cells into a medicament; and
(g) administering the medicament to the subject.

As above, the cell-containing sample of step (a) may be obtained from a subject, e.g. directly from peripheral blood or via leukapheresis, or from a cell source, such as from iPSC cells, to provide iPSC derived Tregs. As also discussed above, the Treg cell may not be genetically engineered and therefore, the transduction/transfection step, step (c), may be removed.

Alternatively viewed, the invention provides a regulatory T cell (Treg) or population of Tregs for use in immunomodulation of a subject, wherein said Treg or population of Tregs is cultured with one or more glial cell line-derived neurotrophic factor (GDNF) family ligands (GFLs) prior to administration to said subject.

In a further aspect, the invention provides a method of producing an immunomodulatory medicament, the method comprising the steps of:
(a) culturing ex vivo a regulatory T cell (Treg) or population of Tregs with one or more glial cell line-derived neurotrophic factor (GDNF) family ligands (GFLs); and
(b) formulating the Treg or population of Tregs into a medicament.

Again, any of the above method steps of generating a Treg cell may be used prior to step (a) of the method of producing an immunomodulatory medicament.

As discussed above, Adoptive Cell Therapy (ACT), i.e. adoptive cell transfer of immune cells (e.g. Tregs, such as engineered Tregs) is an attractive approach for generating desirable immune responses, i.e. to modulate the immune system, particularly to suppress or prevent an unwanted immune response, known as “immunomodulation”. The Tregs of the invention may achieve immunomodulation of a subject by inducing, amplifying, attenuating, or preventing immune responses to prevent or treat a disease. ACT is a form of immunotherapy. Therefore, in other words, the invention provides a method of immunotherapy for a subject in need thereof comprising the steps of:
(a) culturing a regulatory T cell (Treg) or a population of Tregs ex vivo with one or more glial cell line-derived neurotrophic factor (GDNF) family ligands (GFLs);
(b) formulating the Treg or population of Tregs into a medicament; and
(c) administering the medicament to the subject.

Similarly, any of the above method steps of generating a Treg cell may be used prior to step (a) of the method of immunotherapy.

The methods described herein may comprise a further step of cryopreserving the medicament after the formulation step, i.e. the medicament comprising the Treg or population of Tregs may be cryopreserved. In some embodiments, the methods may additionally comprise thawing the medicament prior to the administration step. The medicament of the invention may be administered to a patient immediately after/during thawing and without further expansion.

In some aspects, the Treg or population of Tregs may be cryopreserved and thawed prior to being cultured (i.e. contacted) with the one or more GFLs.

The terms “cryopreservation”, or “cryopreserving” as used herein mean to freeze the Tregs, Treg populations or medicaments under conditions where cells remain viable (e.g. during freezing and after any subsequent step of thawing). Viability of cells can be measured by any well-known method of the art, for example flow cytometry using a live/dead stain (e.g. LIVE/DEAD™ Fixable Near-IR-Dead Cell Stain (Thermofisher). Typically, at least 50%, 60%, 70%, 80%, 90% or 95% of cells will remain viable during and after cryopreservation. Viability thus refers to live cells. A skilled person will appreciate that cryopreservation may allow cells to remain viable due to the application of one or more conditions which can effectively stop cell death and which can maintain the structure of the cell (for example, the use of a particular temperature, freezing/thawing rate and/or cryopreservant). Such conditions are known in the art.

The Tregs of the invention may be autologous to the subject to be treated, i.e. the Tregs for use in the preparation of the medicament may be obtained from the subject to be treated. Thus, the method of the invention may comprise a step of obtaining Tregs from the subject to be treated for use in the preparation of the medicament comprising Tregs. Any suitable method for isolating Tregs from the blood or a blood sample of the subject may be used, e.g. leukapheresis.

In a particular embodiment, the invention may comprise a step of performing leukapheresis on the subject to isolate Treg cells for use in the preparation of the medicament before the step of culturing the Treg cells with one or more GFLs, such as GDNF.

Suitably, the Treg of the invention is isolated from peripheral blood mononuclear cells (PBMCs) obtained from a subject. Suitably, the subject from whom the PBMCs are obtained is a mammal, preferably a human. Suitably, the subject to be treated is a mammal, preferably a human. The cell may be generated ex vivo either from a patient's own peripheral blood (1st party), or in the setting of a haematopoietic stem cell transplant from donor peripheral blood (2nd party), or peripheral blood from an unconnected donor (3rd party).

In one example, the immunomodulatory methods of the present invention may ameliorate immunopathology and re-establish tolerance in inflammatory and autoimmune conditions. It will be appreciated that numerous conditions and disorders that involve an immunopathology or require control or management of the immune system may benefit from administration of a medicament comprising Tregs as described herein. In particular, it has been determined that administration of Tregs to transplant recipients may help to treat and/or prevent rejection of the transplant and/or induce operational immune tolerance in a transplant recipient. Thus, the method of immunomodulation of the invention may find particular utility in the transplant setting, i.e. the subject to be treated may be a transplant recipient. Therefore, in one embodiment, the method of immunomodulation may be for treating and/or preventing rejection of a transplant, e.g. treating and/or preventing cellular and/or humoral transplant rejection. Preferably, the transplant is a solid organ or part thereof or a graft. In further embodiments, the method of immunomodulation is for inducing operational tolerance in a transplant recipient (i.e., the maintenance of normal graft function in the absence of immunosuppressive drugs).

In still further embodiments, the method of immunomodulation is for treating and/or preventing graft-versus-host disease (GvHD).

The transplant may be from a living or deceased donor. Typically, the transplant is an allograft or allotransplantation, i.e. a transplant of an organ or tissue between two genetically non-identical members of the same species. However, the transplant may be a xenograft or xenotransplantation, i.e. a transplant of an organ or tissue from one species to another, e.g. a donor porcine heart valve transplanted into a human recipient.

The transplant may be a solid organ (i.e. a whole solid organ or a part thereof) or a graft. In particular, the solid organ may be a liver, kidney, heart, lung, pancreas, intestine or stomach. The intestine typically is small intestine or a part thereof. Thus, the transplant may be a part of a solid organ, such as a portion of a liver (e.g. a portion of the right lobe, such as about 50-70% of the liver of a living donor), a heart valve or a lung lobe. In some embodiments, the transplant is a liver transplant, e.g. a whole liver or a portion of a liver.

A graft typically refers to tissues or cells, i.e. a tissue or cell transplant. In particular, the tissue may be a vascularized composite tissue, skin, a cornea, a blood vessel, a muscle, a heart valve or a bone (e.g. an arm or leg bone). The cells may be islet of Langerhans cells (pancreas islet cells), bone marrow or adult stem cells.

A vascularized composite tissue graft refers to a graft that is composed of multiple different tissues that are transplanted together as a single unit. A typical example is a hand graft, which consists of muscles, skin, bone, vessels, and nerves. Thus, in some embodiments the transplant is a limb, such as a hand or foot.

Rejection of a transplant or transplant rejection refers to immune-mediated rejection of the transplant (i.e. allograft). Rejection may be hyperacute rejection, acute rejection or chronic rejection. Rejection may result in numerous symptoms including abnormal organ function, malaise, anorexia, muscle ache, low fever, increase in white blood count, and graft-site tenderness. In a representative embodiment, liver rejection may present as elevated levels of markers, such as AST, ALT, GGT; abnormal liver function values such as prothrombin time, ammonia level, bilirubin level, albumin concentration; and abnormal blood glucose. Physical symptoms associated with liver transplant rejection may include encephalopathy, jaundice, bruising and bleeding tendency.

Hyperacute rejection is caused by preformed anti-donor antibodies. It is characterized by the binding of these antibodies to antigens on donor tissue, e.g. vascular endothelial cells. Complement activation is involved and the effect is usually rapid and profound, with rejection occurring within minutes to hours after the transplant procedure.

Acute rejection is mediated by T cells and involves direct cytotoxicity and cytokine mediated pathways. Acute rejection is the most common form of rejection and the primary target of immunosuppressive agents. Acute rejection is usually seen within days or weeks of the transplant.

Chronic rejection is the presence of any sign and symptom of rejection after one year. The cause of chronic rejection is still unknown, but an acute rejection is a strong predictor of chronic rejections.

Thus, the method of treating and/or preventing rejection of a transplant may be for treating and/or preventing acute or chronic rejection of a transplant, particularly an acute rejection of a transplant (e.g. liver transplant). Alternatively viewed, the method of treating and/or preventing rejection of a transplant may be for treating and/or preventing cellular transplant rejection, i.e. transplant rejection mediated by T cells.

In view of the potential utility of Tregs to suppress inflammation and autoimmune responses, it will be appreciated that the invention extends to the treatment of subjects with inflammatory or autoimmune diseases or conditions. While this includes the treatment of transplant recipients (e.g. liver transplant recipients), who may suffer from undesired inflammatory responses (e.g. cell mediated transplant rejection), it is not limited to this aspect.

Thus, in some embodiments, the subject to be treated has an inflammatory or autoimmune disease or condition. Alternatively viewed, the invention provides a method of treating an inflammatory or autoimmune disease or condition in a subject in need thereof, i.e. in some embodiments, the method of immunomodulation is for treating and/or preventing an inflammatory or autoimmune disease or condition. Thus, in some embodiments, the subject is not a transplant recipient and/or is not on an immunosuppressive therapy.

Thus, the disease, condition or disorder to be treated may be an inflammatory or autoimmune disease, condition or disorder. Thus, the invention may find utility in treating and/or preventing (e.g. reducing the risk of) an inflammatory or an autoimmune disease or disorder. The disease may be chronic or acute, preferably chronic. An inflammatory disorder is any condition associated with unwanted inflammation or with an increase in inflammation. An inflammatory condition or disorder may include any condition or disorder in which inflammatory cells are found, such as after trauma or stroke.

The inflammatory or autoimmune disease may be a neurodegenerative disease, e.g. amyotrophic lateral sclerosis (ALS), Alzheimer's disease, Parkinson's disease or multiple sclerosis. Alternatively, the autoimmune disease may be associated with the gastrointestinal tract (e.g. large and/or small intestine), skin, lung or liver. In one aspect, the inflammatory disease is inflammatory bowel disease. In another aspect, the inflammatory disease is transplant rejection (e.g. solid organ transplant rejection, such as liver transplant rejection). In a further aspect, the inflammatory disease may be stroke.

Thus, the inflammatory or autoimmune disease or disorder may be selected from neurodegenerative disease (including ALS, Alzheimer's, Parkinson's disease), stroke, inflammatory bowel disease (including inflammation of the gastrointestinal tract, for example, Crohn's Disease (CD) and Ulcerative Colitis (UC)), diabetes (e.g. type I diabetes), transplant rejection (e.g. organ or graft rejection), rheumatoid arthritis, multiple sclerosis, systemic lupus erythematosus (SLE), atherosclerosis, asthma (e.g. allergic asthma), rhinitis (e.g. allergic rhinitis), graft versus host disease, inflammatory lung disease (e.g. COPD), inflammatory skin disease (such as psoriasis, eczema and dermatitis) and inflammatory liver disease.

The Tregs and medicaments comprising the Tregs of the present invention may have particularly utility in treating neurodegenerative diseases. Thus, in some embodiments, the invention may find utility in treating or preventing (e.g. reducing the risk of) neuroinflammation or an associated disease or disorder. The neuroinflammation may be chronic or acute, preferably chronic. The neuroinflammation may be neuroinflammation of the central or peripheral nervous system, preferably the central nervous system.

The Treg cells of the present invention may be administered to a subject with a neuroinflammatory disease in order to lessen, reduce, or improve at least one symptom of disease such as muscle weakness, muscle twitches, stiff muscles, muscle wasting, cognitive decline, dementia, behavioural changes, pain and/or fatigue. The at least one symptom may be lessened, reduced, or improved by at least 10%, at least 20%, at least 30%, at least 40%, or at least 50%, or the at least one symptom may be completely alleviated.

The Treg cells may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the Treg cells are not administered, or progression of the disease may be completely stopped.

Suitably, where the subject to be treated is suffering from a neurological disease, disorder or injury, the neurological disease, disorder or injury may be selected from amyotrophic lateral sclerosis (ALS), frontotemporal dementia (FTD), progressive supranuclear palsy (PSP), Parkinson's disease, Alzheimer's disease, Huntington's disease, multiple sclerosis, vascular dementia, mixed dementia, Creutzfeldt-Jakob disease, Chronic Inflammatory Demyelinating Polyneuropathy (CIDP), Taupathy disease, Nasu-Hakola disease, central nervous system lupus, dementia with Lewy bodies, Multiple System Atrophy (Shy-Drager syndrome), progressive supranuclear palsy, cortical basal ganglionic degeneration, acute disseminated encephalomyelitis, seizures, spinal cord injury, traumatic brain injury (e.g. ischemia and traumatic brain injury), depression and autism spectrum disorder. In particular, the neurological disease, disorder or injury may be selected from amyotrophic lateral sclerosis (ALS), frontotemporal dementia (FTD), progressive supranuclear palsy (PSP), Parkinson's disease, Alzheimer's disease, Huntington's disease or multiple sclerosis.

In one embodiment, the subject may have ALS. “ALS” or amyotrophic lateral sclerosis (also known as motor neuron disease or Lou Gehrig's disease) as described herein is a progressive neurodegenerative disorder involving primarily motor neurons in the cerebral cortex, brainstem and spinal cord. It is a a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

There are several ALS and ALS-like syndromes, all of which may be treated by the cells, cell populations, medicaments or combination therapies of the present invention. These include, for example, sporadic ALS, genetically-determined (familial, hereditary) ALS, Primary Lateral Sclerosis (PLS), Progressive Muscular Atrophy (PMA), ALS-Plus syndromes, ALS with Laboratory Abnormalities of Uncertain Significance, ALS-mimic syndromes (including post-poliomyelitis syndrome, multifocal motor neuropathy with or without conduction block, endocrinopathies, especially hyperparathyroid or hyperthyroid states, lead intoxication, infection and paraneoplastic syndromes) (Brooks et al., 2000, ALS and other motor neuron disorders, 1, 293-299). The cell, cell population, medicament or combination therapy of the invention may be for use in treating or preventing sporadic ALS, familial ALS, PLS, PMA, ALS-Plus syndromes, ALS with Laboratory Abnormalities of Uncertain Significance, or ALS-mimic syndromes. Preferably, the cell, cell population, medicament or combination therapy of the present invention may be for use in treating or preventing sporadic or familial ALS.

In another embodiment, the subject may have Parkinson's disease. “Parkinson's disease”, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems. The cell, cell population, medicament or combination therapy of the present invention may be for use in treating or preventing Parkinson's disease.

In another aspect, the invention provides a method of improving survival of a regulatory T cell (Treg) or a population of Tregs after administration to a subject, comprising culturing the Treg or population of Tregs with one or more glial cell line-derived neurotrophic factor (GDNF) family ligands (GFLs) prior to administration of said cells.

The methods of the present invention may be performed ex vivo. As used herein, an “ex vivo” method is any method or process that takes place outside the living body. Therefore, the term “ex vivo” as described herein includes any in vitro method or process. For example, an ex vivo method of the present invention may be a method performed with Tregs that have been obtained from a patient's own blood (i.e., directly from a blood vessel or via leukapheresis), or a method performed with Tregs that have been obtained from other sources, such as from cell lines, stem cells such as induced pluripotent stem cells (IPSCs), umbilical cord blood etc.

As mentioned above, the Treg of the present invention may be derived from a patient, e.g. a subject to be treated. Thus, the Treg cell population may be an ex vivo patient-derived cell population. As described in more detail herein, the cell may have been removed from a subject, optionally transduced or transfected ex vivo with a vector to provide an engineered cell, and expanded and formulated into a medicament prior to administration to the subject. Alternatively, the Treg may be a donor cell, for transfer to a recipient subject, or from a cell line, e.g. a Treg cell line. The cell may further be a pluripotent cell (e.g. an iPSC) which may be differentiated to a Treg prior to formulation in to a medicament.

Thus, the Treg cells (e.g. Treg cell population) of the present invention may be allogenic or autologous to the subject to be treated.

The term “medicament” refers to a pharmaceutical composition and these terms may be used interchangeably herein. Thus, a medicament is a composition that comprises or consists of a therapeutically effective amount of a pharmaceutically active agent. In the context of the present application the term “medicament” typically refers to a composition that comprises or consists of a therapeutically effective amount of a Treg cell or Treg cell population described herein. It will be appreciated that a “medicament comprising Tregs” may alternatively be viewed as a “Treg composition”.

A medicament or pharmaceutical composition preferably includes a pharmaceutically acceptable carrier, diluent or excipient (including combinations thereof). Acceptable carriers or diluents for therapeutic use are well-known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The medicaments and pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” it is meant that the formulation is sterile and pyrogen free. The carrier, diluent, and/or excipient must be “acceptable” in the sense of being compatible with the pharmaceutically active agent (e.g. Treg) and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

In a further embodiment, the invention provides a culture medium suitable for culturing a regulatory T cell (Treg) or a population of Tregs comprising one or more glial cell-line derived neurotrophic factor (GDNF) family ligands (GLFs). In particular, the GFL may be GDNF and thus the invention encompasses a culture medium comprising GDNF. In one embodiment, the culture media may not comprise any other neurotrophic factors. In particular, the culture media may not comprise BDNF and CNTF or BDNF and IGF. Culture media suitable for culturing Tregs are well known in the art. Examples of such media are XVIVO™ and TexMACS™. The GFL, such as GDNF, may be included in the culture medium at a concentration of about 1 ng/ml to about 40 ng/ml. For example, the concentration may be about 1.25, 2.5, 5, 10, 20 or 40 ng/ml. For example, the concentration may be from about 1-20, 5-15, 7.5-12.5 or 9-11 ng/ml. In particular, the concentration of the GFL may be 10 ng/ml. The culture medium may also contain other factors suitable for culturing Tregs, such as human AB serum and/or IL-2.

Also provided is a kit comprising a culture media and one or more GFLs, such as GDNF. Preferably said kit is for use in the methods and uses as described herein, e.g. the therapeutic methods as described herein. Preferably said kit comprises instructions for the use of the kit components.

The present invention further provides a combination therapy or product comprising one or more GFLs, such as GDNF, and a medicament comprising Tregs. The components of the combination therapy or product may be for concurrent, or separate and sequential, use in immunomodulation of a subject. For example, the one or more GLFs, e.g., GDNF, may be administered to the subject prior to, concurrently with, or subsequently to administration of the medicament.

The medicament and/or combination therapy or product may be administered in a manner appropriate for the therapeutic purpose described herein, e.g. for use in immunomodulation of a subject.

While the quantity and frequency of administration of the medicament and/or combination therapy or product will be determined by such factors as the condition of the subject, and the type and severity of the subject's disease, as shown herein, the inventors have determined that culturing Tregs with one or more GFLs advantageously enhances their persistence and thus reduces the time required to obtain a therapeutic dose of cells. Thus, in some embodiments, the subject is administered a single dose of the medicament. The medicament and combination therapy or product may be formulated accordingly.

The medicament and/or combination therapy or product can be administered via any suitable means. For instance, the medicament comprising Tregs and/or the combination therapy comprising one or more GFLs, such as GDNF, and the medicament comprising Tregs may be administered parenterally, for example, intravenously, or they may be administered by infusion techniques. The medicament and/or combination therapy or product may be administered in the form of a sterile aqueous solution which may contain other substances, for example, enough salts or glucose to make the solution isotonic with blood. The aqueous solution may be suitably buffered (preferably to a pH of from 3 to 9). The medicament and/or combination therapy may be formulated accordingly. The preparation of suitable parenteral formulations under sterile conditions is readily accomplished by standard pharmaceutical techniques well-known to those skilled in the art.

The medicament comprising Tregs and/or the combination therapy or product may be formulated in infusion media, for example sterile isotonic solution. The medicament or combination therapy or product may be enclosed in ampoules, disposable syringes or multiple dose vials made of glass or plastic.

The medicament and/or combination therapy or product may further comprise one or more active agents.

Depending upon the disease and subject to be treated, as well as the route of administration, the medicament comprising Tregs may be administered at varying doses (e.g. measured in cells/kg or cells/subject). The physician in any event will determine the actual dosage which will be most suitable for any individual subject and it will vary with the age, weight and response of the particular subject. Typically, however, for Tregs, doses of 5×10⁷ to 3×10⁹ cells, or 2×10⁸ to 2×10⁹ cells per subject may be administered.

The cells may be appropriately modified for use in a medicament. For example, Tregs may be cryopreserved and thawed at an appropriate time, before being infused into a subject.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Materials and methods

### Assay for Assessing the Effect of Various Growth Factors on T Cell Survival

A mixed population of non-transduced (NTD) Treg and non-Treg cells were polyclonally expanded for 14 days and then rested for 24 hours in Xvivo with 5% human AB serum (Xvivo-5). The following day cells were counted, resuspended in Xvivo-5 and seeded in wells of a 96-well round-bottomed plate. Cells were stimulated for 6 days with anti-CD3/anti-CD28 beads in the presence of IL-2 alone, or in combination with 10 ng/ml of brain-derived neurotrophic factor (BDNF), glial cell-line derived neurotrophic factor (GDNF), ciliary neurotrophic factor (CNTF), insulin growth factor (IGF), BDNF plus GDNF plus CNTF (all 10 ng/ml), or BDNF plus GDNF plus IGF (all 10 ng/ml). Fresh IL-2 was supplemented on day 4 and the cells were cultured for an additional 2 days. Flow cytometric analysis was performed on day 6 using viability dye and staining for CD4 and FOXP3.

### Assay for Assessing the Effect of GDNF on the Survival of a Highly Pure Population of Tregs

Tregs were transduced with a nucleic acid that encodes a suicide moiety comprising a CD34 marker (which is recognised by the monoclonal antibody QBEnd10), the transcription factor FOXP3 and a CAR. This construct (CI) is described in WO2022/043483. The CAR-transduced Tregs were polyclonally expanded for 14 days and then rested for 24 hours in Xvivo with 5% human AB serum (Xvivo-5). The next day the cells were purified by magnetic-activated cell sorting (MACS), using the QBEnd10 antibody, to enrich for the CAR-Treg expressing cells. These cells were then resuspended in Xvivo-5 and seeded in wells of a 96-well round-bottomed plate. Cells were stimulated for 6 days with anti-CD3/anti-CD28 beads in the presence of IL-2 alone, or in combination with different concentrations of glial cell-line derived neurotrophic factor (GDNF) achieved by serial dilution (1.25, 2.5, 5, 10, 20, 40 or 80 ng/ml GDNF). Alternatively, cells were rested in the presence of the same factors and in the absence of anti-CD3/anti-CD28 beads. Fresh IL-2 was supplemented on day 4 and the cells were cultured for an additional 2 days. Flow cytometric analysis was performed on day 6 using viability dye and staining for CD4 and FOXP3.

### Results

### Example 1

### Assessing the Effect of Various Growth Factors on T Cell Survival

As described in the materials and methods section, a mixed population of non-transduced (NTD) Treg and non-Treg cells from 3 healthy donors (n=3) were expanded for 14 days and then rested for 24 h in Xvivo-5 prior to stimulation and culture in Xvivo-5 alone (control), or with BDNF, GDNF, CNTF, IGF, BDNF plus GDNF plus CNTF or BDNF plus GDNF plus IGF.

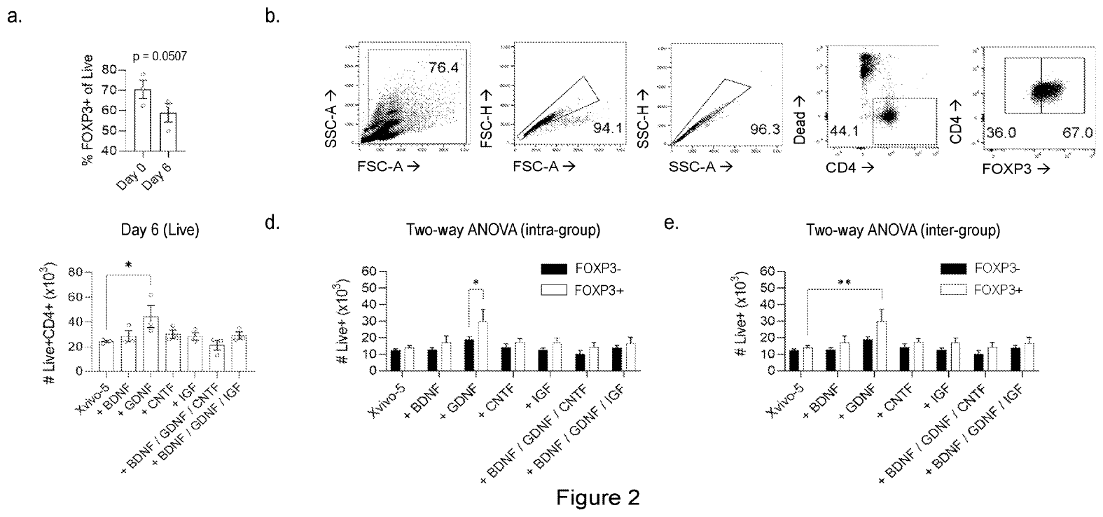

FIG. 2(c) shows that GDNF alone significantly increases the number of Live+CD4+ cells in comparison to Xvivo-5 control, whereas the other growth factors, alone or in combination, have no effect. FIGS. 2(d) and(e) show that this effect is specific for FOXP3+ cells (i.e., Tregs) and not FOXP3− cells (i.e., T effectors). FIG. 2(d) shows that only in the GDNF treatment group is the number of FOXP3+ cells (white bars) significantly higher than the number of FOXP3− cells (black bars), and FIG. 2(e) shows that only in the FOXP3+group (white bars) is the number of cells significantly higher after GDNF treatment compared to the Xvivo-5 control.

Data are from one independent experiment conducted with cells from 3 healthy blood donors (n=3).

### Example 2

### Assessing the Effect of GDNF on the Survival of a Highly Pure Population of Tregs

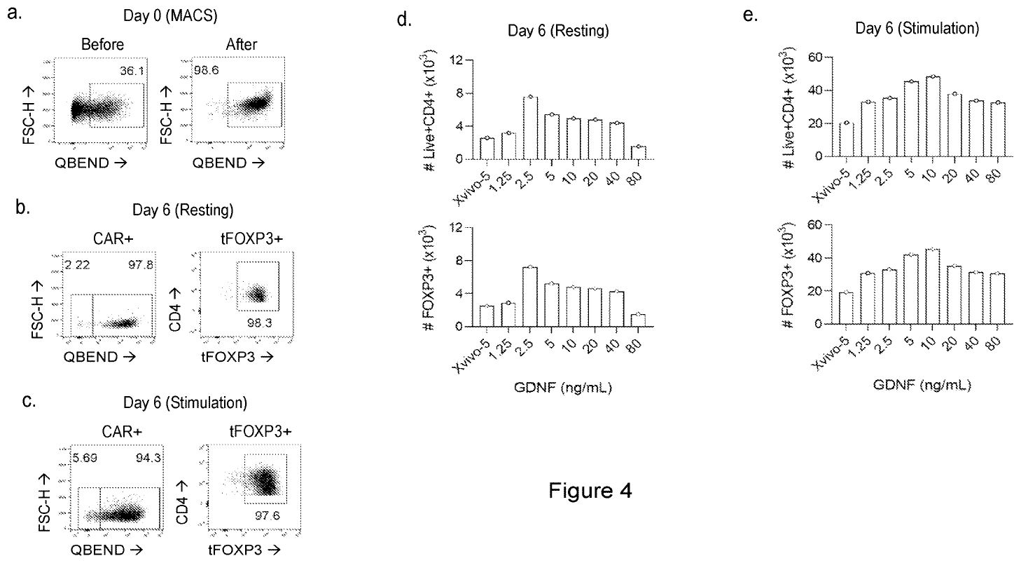

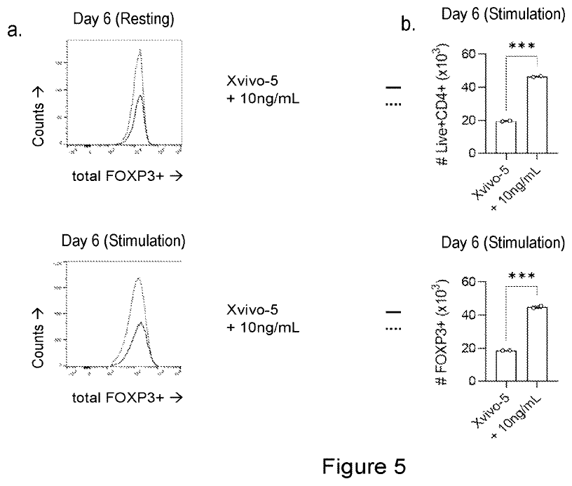

As described in the materials and methods section, CAR-transduced Treg cells were expanded for 14 days and then rested for 24 h in Xvivo-5, prior to enriching for CAR-Treg cells. Enriched cells were either stimulated or unstimulated and cultured in Xvivo-5 alone (control), in different concentrations of GDNF (1.25, 2.5, 5, 10, 20, 40 or 80 ng/ml) (FIG. 4), or in a single concentration of GDNF (10 ng/ml) (FIG. 5).

FIGS. 4(d) and (e), and FIG. 5(b) show that GDNF significantly increases the number of CAR-Treg cells at rest (FIG. 4(d)), and at stimulation conditions (FIG. 4(e) and FIG. 5(b)), thereby resulting in a higher number of FOXP3+ CAR-Treg cells in comparison to Xvivo-5 control. FIG. 4(e) further shows that under stimulation conditions, GDNF acts in a concentration-dependent manner to enhance Treg persistence and 10 ng/ml of this factor yields the optimum effect.

Data are from one independent experiment with cells from 1 healthy blood donor (n=1).

As shown in FIG. 5, the experiment was repeated with the optimum concentration of GDNF only (10 ng/ml) and the results were the same, i.e., GDNF significantly enhanced the number of Live+CD4+ cells (unpaired t-test, p=0.0002) (FIG. 5(a)), thereby resulting in a significantly higher number of FOXP3+ CAR-Treg cells (unpaired t-test, p=0.0006) (FIG. 5(b)) in comparison to Xvivo-5 control.

Data are from two independent experiments conducted with cells from 1 healthy blood donor (n=1).

1. An ex vivo method of increasing the persistence of a regulatory T cell (Treg) or a population of Tregs, comprising the step of culturing the Treg or population of Tregs with one or more glial cell line-derived neurotrophic factor (GDNF) family ligands (GFLs).

2. An ex vivo method of reducing the expansion time of a regulatory T cell (Treg) or a population of Tregs, comprising the step of culturing the Treg or population of Tregs with one or more glial cell line-derived neurotrophic factor (GDNF) family ligands (GFLs).

3. A method of expanding a regulatory T cell (Treg) or a population of Tregs, comprising the step of culturing said Treg or population of Tregs in cell culture media comprising one or more glial cell line-derived neurotrophic factor (GDNF) family ligands (GFLs).

4. The method of claim 1, wherein the persistence of the Treg or population of Tregs is increased under resting conditions, and/or during stimulation.

5. The method of claim 2, wherein the expansion time is at least 50% less than for cells that have not been cultured with one or more GFLs.

6. A method of immunomodulation of a subject in need thereof comprising the steps of:
(a) culturing a regulatory T cell (Treg) or a population of Tregs ex vivo with one or more glial cell line-derived neurotrophic factor (GDNF) family ligands (GFLs);
(b) formulating the Treg or population of Tregs into a medicament; and
(c) administering the medicament to the subject.

7. A method of producing an immunomodulatory medicament, the method comprising the steps of:
(a) culturing ex vivo a regulatory T cell (Treg) or population of Tregs with one or more glial cell line-derived neurotrophic factor (GDNF) family ligands (GFLs); and
(b) formulating the Treg or population of Tregs into a medicament.

8. The method of claim 6 or 7, further comprising a step of performing leukapheresis on the subject to isolate the Treg or population of Tregs prior to step (a).

9. The method of claim 6, further comprising a step of cryopreserving the medicament after step (b) and thawing the medicament prior to step (c), or the method of claim 7, further comprising a step of cryopreserving the medicament after step (b).

10. The method of any one of claims 6 to 9, wherein the method of immunomodulation or the immunomodulatory medicament is for treating an inflammatory or autoimmune disease or condition.

11. The method of any one of claims 6 to 9, wherein the method of immunomodulation or the immunomodulatory medicament is for treating and/or preventing rejection of a transplant.

12. A method of improving survival of a regulatory T cell (Treg) or a population of Tregs after administration to a subject, comprising culturing the Treg or population of Tregs with one or more glial cell line-derived neurotrophic factor (GDNF) family ligands (GFLs) prior to administration of said cells.

13. The method of any preceding claim, wherein the GFL is selected from GDNF, neurturin (NRTN), artemin (ARTN) or persephin (PSPN).

14. The method of claim 13, wherein GDNF has the sequence of SEQ ID NO:1, NRTN has the sequence of SEQ ID NO:2, ARTN has the sequence of SEQ ID NO:3, PSPN has the sequence of SEQ ID NO:4, or is a truncated form, mutated form or sequence variant thereof, wherein the sequence variant has about 95%, 90%, 85%, 80%, 75% or 70% sequence identity to SEQ ID NO:1, SEQ ID NO:2, SEQ ID NO:3 or SEQ ID NO:4.

15. The method of claim 13, wherein GDNF has the sequence of SEQ ID NO:13, NRTN has the sequence of SEQ ID NO:14, ARTN has the sequence of SEQ ID NO:15, PSPN has the sequence of SEQ ID NO:16, or is a truncated form, mutated form or sequence variant thereof, wherein the sequence variant has about 95%, 90%, 85%, 80%, 75% or 70% sequence identity to SEQ ID NO:13, SEQ ID NO:14, SEQ ID NO:15 or SEQ ID NO:16.

16. The method of any preceding claim, wherein the GFL is GDNF.

17. The method of any preceding claim, wherein the concentration of the GFL is from about 1 ng/ml to about 40 ng/ml.

18. The method of any preceding claim, wherein the concentration of the GFL is about 1.25 ng/ml, 2.5 ng/ml, 5 ng/ml, 10 ng/ml, 20 ng/ml or 40 ng/ml, preferably wherein the concentration is about 10 ng/ml.

19. The method of any preceding claim, wherein the Treg or population of Tregs is cultured with a one-off, single dose of the GFL.

20. The method of any preceding claim, wherein the Treg or population of Tregs is an engineered Treg or population of Tregs.

21. The method of claim 20, wherein the Treg or population of Tregs is engineered to comprise a chimeric antigen receptor (CAR) and/or to express an exogenous FOXP3 polypeptide.

22. The method of any preceding claim, wherein the Treg or population of Tregs has been cryopreserved and thawed prior to being cultured with the one or more GFLs.

23. A culture medium suitable for culturing a regulatory T cell (Treg) or a population of Tregs comprising one or more glial cell-line derived neurotrophic factor (GDNF) family ligands (GLFs).

24. Use of one or more glial cell line-derived neurotrophic factor (GDNF) family ligands (GFLs) for increasing the persistence of a regulatory T cell (Treg) or a population of Tregs.

25. Use of one or more glial cell line-derived neurotrophic factor (GDNF) family ligands (GFLs) for reducing the expansion time of a regulatory T cell (Treg) or a population of Tregs.

26. A regulatory T cell (Treg) or population of Tregs for use in immunomodulation of a subject, wherein said Treg or population of Tregs is cultured with one or more glial cell line-derived neurotrophic factor (GDNF) family ligands (GFLs) prior to administration to said subject.

27. A combination therapy or product comprising one or more glial cell line-derived neurotrophic factor (GDNF) family ligands (GLFs) and a medicament comprising regulatory T cells (Tregs), wherein the components of the combination therapy or product are for concurrent, or separate and sequential, use in immunomodulation of a subject.

28. A kit comprising:
(i) one or more glial cell line-derived neurotrophic factor (GDNF) family ligands (GFLs); and
(ii) a culture medium suitable for culturing regulatory T cells (Tregs).

29. The culture medium according to claim 23, the use according to claim 24, 25 or 26, the combination therapy or product according to claim 27 or the kit according to claim 28, wherein the GFL, medicament, subject or immunomodulation is as defined in any one of claims 8 to 11 and 13 to 22.
